SEQUENCE LISTING (61 sequences)

SEQ ID NO: 1 - protein, 57 aa, Synthetic construct
GTMTFQFRNPNFGGNPNNGAFLLNSAQAQNGGELAAKLWANGDETNALSLFQTIIQS

SEQ ID NO: 2 - protein, 57 aa, Synthetic construct
GTMTFQFRNPNFGGNPNNGAFLLNSAQAQNGGELAARLWANGDETNALSLFQTIIQS

SEQ ID NO: 3 - protein, 58 aa, Synthetic construct
GTMTFQFRNPNFGGNPNNGAFLLCSAQAQNGGELAARLWANGDETNALSLFQTIIQSC

SEQ ID NO: 4 - protein, 57 aa, Synthetic construct
GTMTFQFRNPNFGGNPNNGAFLLNSAQAQNAGELAKKLWENGNVNQALSLFQTVIQS

SEQ ID NO: 5 - protein, 57 aa, Synthetic construct
GTMTFQFRNPNFGGNPNNGAFLLNSAQAQNAGELARRLWENGNVNQALSLFQTVIQS

SEQ ID NO: 6 - protein, 58 aa, Synthetic construct
GTMTFQFRNPNFGGNPNNGAFLLCSAQAQNAGELARRLWENGNVNQALSLFQTVIQSC

SEQ ID NO: 7 - protein, 57 aa, Synthetic construct
GTMTFQFRNPNFGGNPNNGAFLLNSAQAQNAAELAAKLWANADETNALSLFQTIIQS

SEQ ID NO: 8 - protein, 57 aa, Synthetic construct
GTMTFQFRNPNFGGNPNNGAFLLNSAQAQNAAELAAKLWANADETNALSLFQTLIQS

SEQ ID NO: 9 - protein, 57 aa, Synthetic construct
GTMTFQFRNPNFGGNPNNGAFLLNSAQAQNAGELAAKLFKKGDLTNALSLFQTVIQS

SEQ ID NO: 10 - protein, 57 aa, Synthetic construct
GTMTFQFRNPNFGGNPNNGAFLLNSAQAQNAGELVEKLFKNGDWTNAISIFQTVIQS

SEQ ID NO: 11 - protein, 57 aa, Synthetic construct
GTMTFQFRNPNFGGNPNNGAFLLNSAQAQNAGELAEKLWRNGDETNALSLFQTVIQS

SEQ ID NO: 12 - protein, 57 aa, Synthetic construct
GTMTFQFRNPNFGGNPNNGAFLLNSAQAQNAGELAEKLWKNGDETNALSLFQTVIQS

SEQ ID NO: 13 - protein, 57 aa, Synthetic construct
GTMTFQFRNPNFGGNPNNGAFLLNSAQAQNAGELAKKLWENGDETNALSLFQTVVQS

SEQ ID NO: 14 - protein, 57 aa, Synthetic construct
GTMTFQFRNPNFGGNPNNGAFLLNSAQAQNAGELAEKLWRNGNESDALSLFQTVIQS

SEQ ID NO: 15 - protein, 57 aa, Synthetic construct
GTMTFQFRNPNFGGNPNNGAFLLNSAQAQNAGELAKKLFENGDKTNALSLFQTVIQS

SEQ ID NO: 16 - protein, 57 aa, Synthetic construct
GTMTFQFRNPNFGGNPNNGAFLLNSAQAQNAGELAKKLWENGDETNALSLFQTVIQS

SEQ ID NO: 17 - protein, 57 aa, Synthetic construct
GTMTFQFRNPNFGGNPNNGAFLLNSAQAQNAGELAKKLWEKGNSEDALALFRTVVQS

SEQ ID NO: 18 - protein, 57 aa, Synthetic construct
GTMTFQFRNPNFGGNPNNGAFLLNSAQAQNAGELAKKLFDNGDMENAMKLFQTVIAS

SEQ ID NO: 19 - protein, 57 aa, Synthetic construct
GTMTFQFRNPNFGGNPNNGAFLLNSAQAQNAGELAEKLWRNGDKDRALALFRTVIQS

SEQ ID NO: 20 - protein, 57 aa, Synthetic construct
GTMTFQFRNPNFGGNPNNGAFLLNSAQAQNAGELADKLWKNGDKDRALSLFQTVIQS

SEQ ID NO: 21 - protein, 57 aa, Synthetic construct
GTMTFQFRNPNFGGNPNNGAFLLNSAQAQNAGELAKKLFDNGDMDRALALFRTVIAS

SEQ ID NO: 22 - protein, 57 aa, Synthetic construct
GTMTFQFRNPNFGGNPNNGAFLLNSAQAQNAGELAKKLFDNGNEEDALALFRTVVAS

SEQ ID NO: 23 - protein, 57 aa, Synthetic construct
GTMTFQFRNPNFGGNPNNGAFLLNSAQAQNAGELAAKLWKKGDEENALKLFRTVVTS

SEQ ID NO: 24 - protein, 57 aa, Synthetic construct
GTMTFQFRNPNFGGNPNNGAFLLNSAQAQNAGELAAKLFKNGNMEDALKLFRTVIAS

SEQ ID NO: 25 - protein, 57 aa, Synthetic construct
GTMTFQFRNPNFGGNPNNGAFLLNSAQAQNAGKVAAILWKNGNKSDALSLFQTVVTS

SEQ ID NO: 26 - protein, 57 aa, Synthetic construct
GTMTFQFRNPNFGGNPNNGAFLLNSAQAQNAGELAAKLFKNGDLTNALSLFQTVVQS

SEQ ID NO: 27 - protein, 57 aa, Synthetic construct
GTMTFQFRNPNFGGNPNNGAFLLNSAQAQNAGELGLKLLRKGDVETALTLFAQVISG

SEQ ID NO: 28 - protein, 57 aa, Synthetic construct
GTMTFQFRNPNFGGNPNNGAFLLNSAQAQNAGELGLKLILKGDLETALKLFAIVIAG

SEQ ID NO: 29 - protein, 57 aa, Synthetic construct
GTMTFQFRNPNFGGNPNNGAFLLNSAQAQNAGELGLKLLRKGDVETALKLFAIVIAG

SEQ ID NO: 30 - protein, 57 aa, Synthetic construct
GTMTFQFRNPNFGGNPNNGAFLLNSAQAQNAGELAKKLYENGLIELALMLFALVIAS

SEQ ID NO: 31 - protein, 57 aa, Synthetic construct
GTMTFQFRNPNFGGNPNNGAFLLNSAQAQNAGELYKKLWDNGEVDKALDLFAKIIAG

SEQ ID NO: 32 - protein, 57 aa, Synthetic construct
GTMTFQFRNPNFGGNPNNGAFLLNSAQAQNAGELGKKLIEKGDLETALKLFAIVIAG

SEQ ID NO: 33 - protein, 57 aa, Synthetic construct
GTMTFQFRNPNFGGNPNNGAFLLNSAQAQNAGEIALRLLKNGKEEEALKTLLVTIAG

SEQ ID NO: 34 - protein, 57 aa, Synthetic construct
GTMTFQFRNPNFGGNPNNGAFLLNSAQAQNAGELAAKLWKKGDETNALSLFQTVVTS

SEQ ID NO: 35 - protein, 57 aa, Synthetic construct; identical to SEQ ID NO: 25

SEQ ID NO: 36 - protein, 57 aa, Synthetic construct
GTMTFQFRNPNFGGNPNNGAFLLNSAQAQNAGELAKKLWEKGDETNALSLFQTVVTS

SEQ ID NO: 37 - protein, 57 aa, Synthetic construct
GTMTFQFRNPNFGGNPNNGAFLLNSAQAQNAGDLAAKLWKKGDETNALSLFQTVVTS

SEQ ID NO: 38 - protein, 57 aa, Synthetic construct
GTMTFQFRNPNFGGNPNNGAFLLNSAQAQNAGELAAKLWKNGNSSDALSLFQTVVTS

SEQ ID NO: 39 - protein, 57 aa, Synthetic construct; identical to SEQ ID NO: 36

SEQ ID NO: 40 - protein, 57 aa, Synthetic construct
GTMTFQFRNPNFGGNPNNGAFLLNSAQAQNAGELAKKLWEKGDSSNALSLFQTVVTS

SEQ ID NO: 41 - protein, 57 aa, Synthetic construct
GTMTFQFRNPNFGGNPNNGAFLLNSAQAQNAGDLAAKLWKNGDETNALSLFQTVVTS

SEQ ID NO: 42 - protein, 57 aa, Synthetic construct; identical to SEQ ID NO: 26

SEQ ID NO: 43 - protein, 57 aa, Synthetic construct; identical to SEQ ID NO: 34

SEQ ID NO: 44 - protein, 57 aa, Synthetic construct
GTMTFQFRNPNFGGNPNNGAFLLNSAQAQNAGELAAKLFNSGDLDRALALFRTVVTS

SEQ ID NO: 45 - protein, 57 aa, Synthetic construct
GTMTFQFRNPNFGGNPNNGAFLLNSAQAQNAGKVAKELYDNGDEKWALLLFRTVVTS

SEQ ID NO: 46 - protein, 57 aa, Synthetic construct
GTMTFQFRNPNFGGNPNNGAFLLNSAQAQNAGKVAAELYKNGDEKNALLLFRTVVAS

SEQ ID NO: 47 - protein, 57 aa, Synthetic construct
GTMTFQFRNPNFGGNPNNGAFLLNSAQAQNAGELAAKLFKNGDMENALALFRTVVTS

SEQ ID NO: 48 - protein, 57 aa, Synthetic construct; identical to SEQ ID NO: 17

SEQ ID NO: 49 - protein, 57 aa, Synthetic construct
GTMTFQFRNPNFGGNPNNGAFLLNSAQAQNAGELAAKLFNKGDEDRALALFRTVVQS

SEQ ID NO: 50 - protein, 57 aa, Synthetic construct
GTMTFQFRNPNFGGNPNNGAFLLNSAQAQNAGELAAKLWKNGDEENALALFRTVVTS

SEQ ID NO: 51 - protein, 57 aa, Synthetic construct
GTMTFQFRNPNFGGNPNNGAFLLNSAQAQNAGELAEKLWRSGDADRALALFRTVVTS

SEQ ID NO: 52 - protein, 57 aa, Synthetic construct
GTMTFQFRNPNFGGNPNNGAFLLNSAQAQNAGELAAKLWKNGNEEDALALFRTVVTS

SEQ ID NO: 53 - protein, 57 aa, Synthetic construct
GTMTFQFRNPNFGGNPNNGAFLLNSAQAQNAGELAAKLFNNGDEDRALALFRTVVQS

SEQ ID NO: 54 - protein, 57 aa, Synthetic construct
GTMTFQFRNPNFGGNPNNGAFLLNSAQAQNAGELAAKLWKKGDEDRALALFRTVVTS

SEQ ID NO: 55 - protein, 57 aa, Synthetic construct
GTMTFQFRNPNFGGNPNNGAFLLNSAQAQNAGELAAKLFNSGDEDRALALFRTVVQS

SEQ ID NO: 56 - protein, 57 aa, Synthetic construct
GTMTFQFRNPNFGGNPNNGAFLLNSAQAQNAGELAAKLYNNGDLDRADATFRTVVQS

SEQ ID NO: 57 - protein, 57 aa, Synthetic construct
GTMTFQFRNPNFGGNPNNGAFLLNSAQAQNAGELAKKLWENGNEEDALALFRTVVTS

SEQ ID NO: 58 - protein, 57 aa, Synthetic construct
GTMTFQFRNPNFGGNPNNGAFLLNSAQAQNAGEIAKQLWEKGDESSAITVATIVLSS

SEQ ID NO: 59 - protein, 262 aa, Escherichia coli
CLTAPPKEAARPTLMPRAQSYKDLTHLPAPTGKIFVSVYNIQDETGQFKPYPASNFSTAVPQSATAMLVTALKDSRWFIPLERQGLQNLLNERKIIRAAQENGTVAINNRIPLQSLTAANIMVEGSIIGYESNVKSGGVGARYFGIGADTQYQLDQIAVNLRVVNVSTGEILSSVNTSKTILSYEVQAGVFRFIDYQRLLEGEVGYTSNEPVMLCLMSAIETGVIFLINDGIDRGLWDLQNKAERQNDILVKYRHMSVPPES

SEQ ID NO: 60 - protein, 30 aa, Synthetic construct
GTMTFQFRNPNFGGNPNNGAFLLNSAQAQN

SEQ ID NO: 61 - protein, 57 aa, Synthetic construct
GTMTFQFRNPNFGGNPNNGAFLLNSAQAQNAGILAAQLWNNGDYDRALSLFIAVVQS

(19) United States
(12) Patent Application Publication
(10) Pub. No.: US 2026/0358293 A1
(43) Pub. Date: Nov. 12, 2026

(54) DE NOVO PORES

(71) Applicants: Oxford Nanopore Technologies PLC, Oxford (GB); The Regents of the University of California, Oakland, CA (US)

(72) Inventors: Elizabeth Jayne Wallace, Oxford (GB); Lakmal Nishantha Jayasingle, Oxford (GB); Richard George Hambley, Oxford (GB); Alistair James Scott, Oxford (GB); Ranga Prabhath Malaviarachchige Rabel, Oxford (GB); Rhys Connor Griffiths, Oxford (GB); Amber Elizabeth Leckenby, Oxford (GB); Pratik Raj Singh, Oxford (GB); Alberto Riera, Oxford (GB); William F. DeGrado, Oakland, CA (US); Lee Schnaider, Oakland, CA (US); Nicholas Polizzi, Oakland, CA (US)

(73) Assignees: Oxford Nanopore Technologies PLC, Oxford (GB); The Regents of the University of California, Oakland, CA (US)

(21) Appl. No.: 19/101,976

(22) PCT Filed: Aug. 9, 2023
(86) PCT No.: PCT/EP2023/072113

§ 371 Date: Feb. 7, 2025

Related U.S. Application Data
(60) Provisional application No. 63/396,539, filed on Aug. 9, 2022.

(51) Int. Cl. G01N 27/447; C07K 14/245; C12Q 1/6869; G01N 33/487

(52) U.S. Cl. CPC G01N 27/44791; C07K 14/245; C12Q 1/6869; G01N 27/4473; G01N 33/48721; C07K 2319/00

(57) ABSTRACT

Aspects of the disclosure relate to protein pore complexes and their uses in analyte detection and characterisation. The disclosure is based, in part, on nanopore complexes formed by CsgG-like pores and one or more auxiliary proteins, which form one or more channel constrictions in the nanopore complex. In some embodiments, the one or more auxiliary protein is a fusion protein. The disclosure further relates to methods for design of auxiliary proteins and production of the nanopore complexes, and for use in molecular sensing and analyte sequencing applications.

### RELATED APPLICATIONS

This application is a national stage filing under 35 U.S.C. § 371 of international application number PCT/EP2023/072113, filed Aug. 9, 2023, which claims the benefit of U.S. application No. 63/396,539, filed Aug. 9, 2022, each of which is herein incorporated by reference in its entirety.

### REFERENCE TO AN ELECTRONIC SEQUENCE LISTING

The contents of the electronic sequence listing (O036670130US01-SEQ-KZM.xml; Size: 56,705 bytes; and Date of Creation: Jan. 31, 2025) are herein incorporated by reference in its entirety.

### BACKGROUND

Two important components of polymer characterization using nanopore sensing are (1) the control of polymer movement through the pore, and (2) the discrimination of the composing building blocks as the polymer is moved through the pore. During nanopore sensing, the narrowest part of the pore forms the constriction, the most discriminating part of the nanopore with respect to the current signatures as a function of the passing analyte. CsgG was identified as an ungated, non-selective protein secretion channel from Escherichia coli (Goyal et al., 2014) and has been used as a nanopore for detecting and characterising analytes. Mutations to the wild-type CsgG pore that improve the properties of the pore in this context have also been disclosed (WO2016/034591, WO2017/149316, WO2017/149317 and WO2017/149318, PCT/GB2018/051191, all incorporated by reference herein in their entireties).

For analytes being polynucleotides, nucleotide discrimination is achieved via passage through such a mutant pore, but current signatures have been shown to be sequence dependent, and multiple nucleotides contributed to the observed current, so that the height of the channel constriction and extent of the interaction surface with the analyte affect the relationship between observed current and polynucleotide sequence. While the current range for nucleotide discrimination has been improved through mutation of the CsgG pore, a sequencing system would have higher performance if the current differences between nucleotides could be improved further.

### SUMMARY

The disclosure relates, in some aspects, to protein pore complexes and their uses in analyte detection and characterisation. The disclosure is based, in part, on nanopore complexes formed by CsgG pores and one or more auxiliary proteins, which form one or more channel constrictions in the nanopore complex. In some embodiments, the one or more auxiliary protein is a fusion protein. As described further in the Examples, it has been surprisingly discovered that auxiliary proteins that confer certain desirable features to CsgG protein nanopores (e.g., modulation of pore width, lengthening of pore lumen, formation of one or more additional constrictions, etc.) can be designed de novo using computer-based structural analysis tools. In some embodiments, the de novo-designed auxiliary proteins (e.g., fusion proteins) form one or more constrictions in the lumen of a CsgG nanopore, and improve discrimination of polymer units as an analyte moves through the nanopore.

Some aspects of the disclosure further relate to methods for design of auxiliary proteins and production of the nanopore complexes, and for use in molecular sensing and nucleic acid sequencing applications.

In some aspects, the disclosure provides a protein nanopore complex comprising a CsgG nanopore comprising a lumen; and a fusion polypeptide comprising a first portion comprising a CsgF protein and a second portion comprising a helix-forming auxiliary protein, wherein the fusion protein is attached to the nanopore.

In some embodiments, the first portion of the fusion protein is attached to the CsgG nanopore. In some embodiments, the first portion of the fusion protein is positioned inside the lumen of the CsgG nanopore. In some embodiments, the first portion of the fusion protein extends outside of the lumen of the CsgG nanopore. In some embodiments, the first portion forms a first constriction region in the lumen of the CsgG nanopore.

In some embodiments, the second portion forms a second constriction region.

In some embodiments, the CsgG nanopore further comprises a constriction region.

In some embodiments, the second portion is not attached to the CsgG nanopore. In some embodiments, the second portion comprises one or more helices (e.g., alpha-helices, etc.).

In some embodiments, each of the helices (e.g., alpha-helices, etc.) of the second portion comprises between 0 and 15 alpha-helical turns. In some embodiments, the second portion comprises a first alpha-helix comprising one to four alpha-helical turns, and a second alpha-helix comprising three to six alpha-helical turns. In some embodiments, the second alpha-helix packs against the first alpha-helix. In some embodiments, the second portion comprises between 1 and 55 amino acid residues. In some embodiments, each of the helices comprises 1-20 amino acid residues having Phi angles ranging from about −45° to −90° and Psi angles ranging from about 0° to −70°. In some embodiments, each of the helices comprises 1-30 amino acid residues having Phi angles ranging from about −45° to −90° and Psi angles ranging from about 0° to −70°.

In some embodiments, the distance (e.g., vertical distance) between the first constriction region and second constriction region ranges from about 5 Å to about 80 Å (e.g., when measured as the distance between the alpha-carbons (Cₐ) of the amino acid residue extending furthest into the lumen of the nanopore forming the first constriction and the amino acid residue extending furthest into the lumen of the nanopore forming the second constriction). In some embodiments, the protein nanopore complex has an axial length greater than 90 Å, optionally wherein the axial length ranges from about 95 Å to about 160 Å.

In some embodiments, the fusion protein is attached to the nanopore by a linker. In some embodiments, the linker comprises a bond, a peptide linker, or a chemical linker. In some embodiments, the linker comprises a bond formed by a Sulfur (VI) fluoride exchange (SuFEx) reaction. In some embodiments, the linker comprises one or more maleimide molecules.

In some embodiments, the fusion protein is cyclised. In some embodiments, the cyclisation comprises one or more side-chain to side-chain cyclisation bonds. In some embodiments, at least one of the side-chain to side-chain cyclisation bonds is a disulfide bond.

In some aspects, the disclosure provides a protein nanopore complex comprising: a CsgG nanopore comprising a lumen and a first constriction region formed within the lumen of the nanopore; and a fusion protein comprising a first portion comprising a CsgF protein and a second portion comprising a helix-forming auxiliary protein, wherein the fusion protein is attached to the nanopore.

In some embodiments, the first portion of the fusion protein is attached to the CsgG nanopore. In some embodiments, the first portion of the fusion protein is positioned inside the lumen of the CsgG nanopore.

In some embodiments, the second portion of the fusion protein is positioned outside the lumen of the CsgG nanopore.

In some embodiments, the first portion forms a second constriction region in the lumen of the CsgG nanopore. In some embodiments, the second portion forms a third constriction region in the lumen of the CsgG nanopore.

In some embodiments, the second portion is not attached to the CsgG nanopore.

In some embodiments, the second portion comprises one or more helices (e.g., alpha-helices, etc.). In some embodiments, each of the helices (e.g., alpha-helices) comprises between 0 and 15 alpha-helical turns. In some embodiments, the second portion comprises between 1 and 54 amino acid residues. In some embodiments, each of the helices comprises 1-36 amino acid residues having Phi angles ranging from about −45° to −90° and Psi angles ranging from about 0° to −70°. In some embodiments, each of the helices comprises 1-36 amino acid residues having Phi angles ranging from about −45° to −90° and Psi angles ranging from about 0° to −70°. In some embodiments, the fusion protein is cyclised. In some embodiments, the cyclisation comprises one or more side-chain to side-chain cyclisation bonds. In some embodiments, the cyclisation comprises one or more side-chain to tail (e.g., C-terminus) cyclisation bonds. In some embodiments, at least one of the cyclisation bonds is a disulfide bond.

In some aspects, the disclosure provides a protein nanopore complex comprising a CsgG nanopore comprising a lumen and a first constriction region formed within the lumen of the nanopore; a first auxiliary protein attached to the CsgG nanopore and forming a second constriction region in the lumen of the nanopore; and a second auxiliary protein attached to the CsgG nanopore or the first auxiliary protein, and forming a third constriction region.

In some embodiments, the first auxiliary protein is positioned inside the lumen of the CsgG nanopore. In some embodiments, the first auxiliary protein comprises a CsgF protein or peptide.

In some embodiments, the second auxiliary protein comprises one or more helices (e.g., alpha-helices, etc.). In some embodiments, each of the one or more helices (e.g., alpha-helices) comprises between 0 and 15 alpha-helical turns. In some embodiments, the second auxiliary protein comprises two alpha-helices.

In some embodiments, one of the alpha-helices comprises between 1 and 6 alpha-helical turns. In some embodiments, one of the alpha-helices comprises between 1 and 10 alpha-helical turns. In some embodiments, one of the alpha-helices comprises three alpha-helical turns, and the other alpha-helix comprises three or four alpha-helical turns. In some embodiments, each of the helices comprises 1-36 amino acid residues having Phi angles ranging from about −45° to −90° and Psi angles ranging from about 0° to −70°. In some embodiments, each of the helices comprises 1-36 amino acid residues having Phi angles ranging from about −45° to −90° and Psi angles ranging from about 0° to −70°

In some embodiments, the second auxiliary protein comprises at least one alpha helix that packs against an alpha-helix of the first auxiliary protein. In some embodiments, the second auxiliary protein comprises between 1 and 55 amino acid residues.

In some embodiments, the distance (e.g., vertical distance) between the first constriction and second constriction ranges from about 20 Å to about 80 Å (e.g., when measured as the distance between the alpha-carbons (Cₐ) of the amino acid residue extending furthest into the lumen of the nanopore forming the first constriction and the amino acid residue extending furthest into the lumen of the nanopore forming the second constriction). In some embodiments, the distance between the second constriction and third constriction ranges from about 5 Å to about 80 Å. In some embodiments, the protein nanopore complex has an axial length greater than 90 Å, optionally wherein the axial length ranges from about 95 Å to about 160 Å.

In some embodiments, the first auxiliary protein and the second auxiliary protein are attached by a linker. In some embodiments, the linker comprises a bond, a peptide linker, or a chemical linker. In some embodiments, the linker comprises a bond formed by a Sulfur (VI) fluoride exchange (SuFEx) reaction. In some embodiments, the linker comprises one or more maleimide molecules. In some embodiments, a linker comprises one or more cyclisation bonds (e.g., a first amino acid of a linker may be covalently or non-covalently attached to a second amino acid of the linker, for example by a crosslinker).

In some embodiments, the first auxiliary protein and the second auxiliary protein comprise one or more side-chain to side-chain cyclisation bonds. In some embodiments, the first auxiliary protein and the second auxiliary protein comprise one or more side-chain to tail (e.g., C-terminus) cyclisation bonds. In some embodiments, at least one of the cyclisation bonds is a disulfide bond.

In some aspects, the disclosure provides a system for characterising a target analyte, the system comprising a protein nanopore complex as described herein inserted into a membrane.

In some embodiments, the system further comprises an electrically-conductive solution in contact with the protein nanopore complex, electrodes providing a voltage potential across the membrane, and a measurement system for measuring the current through the protein nanopore complex.

In some aspects, the disclosure provides a method for characterising a target analyte, the method comprising the steps of contacting a system as described herein with the target analyte; applying a potential across the membrane such that the target analyte enters the lumen formed by the protein nanopore complex; and taking one or more measurements as the target analyte moves with respect to the lumen, thereby characterising the target analyte.

In some embodiments, the target analyte comprises a target polynucleotide.

In some embodiments, the taking one or more measurements comprises measuring the current passing through the continuous channel, wherein the current is indicative of the presence and/or one or more characteristics of the target analyte and thereby detecting and/or characterising the target analyte.

In some embodiments, the target analyte is a polynucleotide and nucleotides in the polynucleotide interact with the first and second (and, optionally, third) constriction regions within the lumen and wherein each of the first, second, (and, optionally, third) constriction regions is capable of discriminating between different nucleotides, such that the overall current passing through the lumen is influenced by the interactions between each of the first, second, and third constriction regions and the nucleotides located at each of the regions.

In some aspects the disclosure provides a method of making a protein nanopore complex, the protein nanopore complex comprising:
(a) a CsgG nanopore comprising a lumen; and
(b) a fusion polypeptide comprising a first portion comprising a CsgF protein and a second portion comprising a helix-forming auxiliary protein, wherein the fusion protein is attached to the nanopore and wherein at least one domain of the fusion polypeptide is designed using a computer generated algorithm.

### BRIEF DESCRIPTION OF DRAWINGS

FIGS. 1A-1C show a workflow for de novo design of a fusion protein. FIG. 1A shows a design workflow using a CsgG nanopore. Wild-type CsgF (residues 1-35; left panel) is shown in orange. Residues 17-30 of wild-type CsgF (red) were chosen as a target to which we searched for a geometrically matching and designable helix that would pack it and project onto the pore to create a new constriction between 10 Å and 30 Å in diameter (cyan) was conducted. The two helices were looped (yellow) and sequence design of the resulting backbone was carried out via Rosetta. FIG. 1B shows helix-helix interactions with symmetry-related partners. FIG. 1C shows a top view of a nonameric CsgG-Fusion protein complex demonstrating the additional constriction achieved by the de novo designed fusion protein.

FIG. 2 shows representative data for prioritization the de novo fusion protein sequences designed using Rosetta. Sequences for experimental validation were chosen based on the lowest energy score and highest PackStat score.

FIGS. 3A-3D show PSIPRED protein secondary structure analysis based on amino acid sequence for the de novo designed fusion proteins. Residues are shaded according to whether they are predicted to be strand, helix and coil, respectively. FIG. 3A shows secondary structure prediction of fusion proteins and the mature sequence of wild-type CsgF. FIG. 3B shows secondary structure analysis for the de novo designed fusion proteins, ONT1 to ONT10. FIG. 3C shows secondary structure analysis for the de novo designed fusion proteins, ONT11 to ONT20.

FIG. 3D shows protein secondary structure analysis for the de novo designed fusion proteins, ONT21 to ONT25.

FIGS. 4A-4C show predicted 3-dimensional structures of alternative sequences for de novo designed fusion proteins. FIG. 4A shows predicted structures for de novo designed fusion proteins ONT1 to ONT10. FIG. 4B shows predicted structures for de novo designed fusion proteins ONT11 to ONT20. FIG. 4C shows predicted structures for de novo designed fusion proteins ONT21 to ONT25.

FIG. 5 shows a representative SDS-PAGE gel analysis of CsgG-only pores and CsgG/fusion protein complexes, where the complexes either comprise the CsgF-del (S31-F119) control or de novo designed fusion proteins, with or without a maleimide crosslinker. The complexes comprising the fusion proteins show a band shift, indicating these samples are pore complexes. Note that the samples were not heated prior to loading onto the gel.

FIG. 6 shows a representative SDS-PAGE gel analysis of CsgG-only pores and CsgG/fusion protein complexes, where the complexes either comprise the CsgF-del (S31-F119) control or de novo designed fusion proteins, with or without a maleimide crosslinker. The pores were broken down to their constituent monomer components upon boiling in the presence of DTT prior to loading onto the gel.

FIG. 7 shows representative ionic current (pA) versus time(s) traces as single stranded DNA translocates through CsgG-only pores. The raw current trace is shown in black lines and the event detected signal is shown in red lines. For each pore, the top row shows the full DNA current trace, whilst the bottom row shows a zoomed in view of the first section of the current trace.

FIG. 8 shows representative ionic current (pA) versus time(s) traces as single stranded DNA translocates through CsgG that comprises del (S31-F119) CsgF peptides, with or without a maleimide crosslinker.

FIG. 9 shows representative ionic current (pA) versus time(s) traces as single stranded DNA translocates through CsgG that comprises a de novo designed fusion protein in the absence of a maleimide crosslinker.

FIG. 10 shows representative ionic current (pA) versus time(s) traces as single stranded DNA translocates through CsgG that comprises the de novo designed fusion protein, plus/minus the maleimide crosslink.

FIG. 11 shows representative ionic current (pA) versus time(s) traces as single stranded DNA translocates through CsgG that comprises a de novo designed fusion protein, with or without a maleimide crosslinker. The fusion protein comprises a K37R mutation along with cysteine residues to form an internal disulfide bond within the peptide, i.e. to cyclise the fusion protein.

FIG. 12 shows representative profiles demonstrating positions within the pore and their contribution to overall changes in ionic current level (“Discrimination”) when a DNA molecule is translocated through the pore. CsgG-only pores (plus/minus Q153C) show one major discrimination peak at position 0.

FIG. 13 shows representative profiles demonstrating positions within the pore and their contribution to overall changes in ionic current level (“Discrimination”) when a DNA molecule is translocated through the pore. Dashed boxes show the region that would be affected by the introduction of a de novo designed fusion protein. CsgG-CsgF-del (S31-F119) pores with or without a maleimide crosslinker show two discrimination peaks. The major discrimination peak at position 0, as seen in CsgG-only pores, and an additional discrimination peak 4-6 nucleotides below the main constriction (position −4 to −6). This additional region of discrimination has less influence on the ionic current compared to the main discrimination peak at position 0.

FIG. 14 shows representative profiles demonstrating positions within the pore and their contribution to overall changes in ionic current level (“Discrimination”) when a DNA molecule is translocated through the pore. Distances within the pore are measured in nucleotide steps relative to the major constriction. Negative values correspond to positions below the main constriction and positive values correspond to positions above the main constriction (CsgG). Dashed boxes show the region that would be affected by the introduction of a de novo designed fusion protein. Complexes comprised of CsgG and de novo designed fusion proteins containing K37R (with or without a maleimide crosslinker; with cyclisation) show three discrimination peaks. The major discrimination peak at position 0, as seen in CsgG-only pores, and additional peaks at positions −6 and −9. The peak at position-9 corresponds to the expected constriction produced by the de novo designed fusion protein when folded in the correct orientation.

FIG. 15 shows an example of two proteins connected by a maleimidopropionic acid linker.

FIG. 16 shows examples of pore proteins and auxiliary (e.g., fusion proteins) functionalised with reactive modifiers, such as thiol modifiers.

FIG. 17 shows representative ionic current (pA) versus time(s) traces as single stranded DNA translocates through CsgG that comprises a de novo designed fusion protein (SEQ ID NO: 61), with (bottom two traces) or without (top two traces) a maleimide crosslinker. The raw current trace is shown in black lines and the event detected signal is shown in red lines. For each pore, the top row shows the full DNA current trace, whilst the bottom row shows a zoomed in view of the first section of the current trace.

FIG. 18 shows representative profiles demonstrating positions within the pore and their contribution to overall changes in ionic current level (“Discrimination”) when a DNA molecule is translocated through the pore. Distances within the pore are measured in nucleotide steps relative to the major constriction. Negative values correspond to positions below the main constriction and positive values correspond to positions above the main constriction (CsgG). Dashed boxes show the region that would be affected by the introduction of a de novo designed fusion protein. Complexes comprised of CsgG and de novo designed fusion proteins (SEQ ID NO: 61) with (bottom profile) or without (top profile) a maleimide crosslinker; both without cyclisation) show three discrimination peaks. The major discrimination peak at position 0, as seen in CsgG-only pores, and additional peaks at positions −5 and −11. The peak at position-11 corresponds to the expected constriction produced by the de novo designed fusion protein when folded in the correct orientation.

FIG. 19 shows the structure and size of the wild-type CsgG pore from Escherichia coli strain K12 (the databank accession code for this structure is 4UV3). The distances shown are measured from backbone to backbone of the amino acids forming the pore structure. The CsgG pore is a tightly interconnected symmetrical nonameric pore that resembles a crown. The overall height is 98 Å, and the largest outer diameter is 120 Å. It defines a central channel and consists of three parts: (A) the cap region, (B) the constriction region and (C) the transmembrane beta barrel region. Cap axial length, or height, is 39 Å. It has an inner diameter of 43 Å and a 66 Å mouth. The beta barrel has 36 strands, an axial length of 39 Å and inner diameter of 55 Å. Transition between pore cap and beta barrel is sharp, being the constriction located among them, at the level of the predicted lipid-aqueous interface. The constriction is approximately 18.5 Å in diameter and exhibits a length of 20 Å along the axis of the channel.

### DETAILED DESCRIPTION

Aspects of the disclosure relate to compositions and methods for characterizing analytes using nanopore-based systems. The disclosure is based, in part, on protein nanopore complexes formed by a CsgG pores and one or more auxiliary proteins, which form one or more channel constrictions in the nanopore complex. In some embodiments, the one or more auxiliary protein is a fusion protein. As described further in the Examples, it has been surprisingly discovered that auxiliary proteins that confer certain desirable features to CsgG nanopores (e.g., modulation of pore width, lengthening of pore lumen, formation of one or more additional constrictions, etc.) can be designed de novo using computer-based structural analysis tools. In some embodiments, the de novo designed auxiliary proteins (e.g., fusion proteins) form one or more additional constrictions in the lumen of a CsgG pore, and improve discrimination of polymer units as an analyte moves through the nanopore.

### Auxiliary Proteins

A protein nanopore complex (also referred to interchangeably as a protein pore complex) as described by the disclosure may include one or more auxiliary proteins. As used herein, the terms “peptide”, “polypeptide” or “protein” are used interchangeably herein and refer to two or more amino acids linked together by a peptide bond. In some embodiments a protein (also referred to as a polypeptide or peptide) comprises between 2 and 2000 amino acids. In some embodiments, a protein comprises between 2 and 10 amino acids, 2 and 25 amino acids, 2 and 50 amino acids, 2 and 100 amino acids, 2 and 500 amino acids, or 2 and 1000 amino acids (or any number of amino acids therebetween, for example 2, 3, 4, 5, 6, 7, 8, 9, 10, 15, 20, 25, 30, 35, 40, 45, 50, 75, 100, 250, 500, 750, 1000 amino acids, etc.). In some embodiments, a protein comprises more than 2000 amino acids. In some embodiments, a peptide, polypeptide, or protein is synthetic in origin (e.g., not present in nature, for example not naturally expressed in any living organism). In some embodiments, a peptide, polypeptide, or protein is naturally occurring (e.g., is naturally expressed in a living organism that has not been genetically modified to express the peptide, polypeptide, or protein). In some embodiments, a peptide, polypeptide, or protein may be expressed naturally by an organism. In some embodiments, a peptide, polypeptide, or protein is heterologously expressed by an organism (e.g., an organism genetically modified to express the peptide, polypeptide, or protein). In some embodiments, a peptide, polypeptide, or protein is chemically synthesized (e.g., by in vitro transcription, peptide synthesis, etc.). A peptide, polypeptide, or protein may comprise one or more naturally-occurring amino acids (L-amino acids, D-amino acids, etc.), one or more non naturally-occurring amino acids (e.g., radiolabeled amino acids, non-canonical amino acids, unnatural amino acids, etc.), or a combination of one or more naturally-occurring amino acids and one or more non naturally-occurring amino acids.

In some embodiments, an auxiliary protein is a fusion protein. The term “fusion protein” refers to a naturally occurring, synthetic, semi-synthetic or recombinant single protein molecule that comprises all or a portion of two or more heterologous polypeptides (e.g., polypeptides that are heterologous with respect to one another) joined by peptide bonds. In some embodiments, a fusion protein comprises all or a portion of at least 2, 3, 4, 5, 6, 7, 8, 9, or 10 heterologous polypeptides joined by peptide bonds. As used herein “a portion of the peptide” refers to 2 or more amino acids of the peptide. In some embodiments, a portion of the peptide comprises at least: 5, 10, 20, 30, 50, or 100 amino acids (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids), either consecutive or with gaps, of the complete amino acid sequence of the peptide, or the full amino acid sequence of the peptide. The portions of a fusion protein may be arranged in any suitable manner (e.g., C-terminal to N-terminal, N-terminal to C-terminal, C-terminal to C-terminal, N-terminal to N-terminal, etc.). In some embodiments, a C-terminal end of a first portion may be joined (e.g., connected) to an N-terminal end of a second portion. Portions of a fusion protein may be joined directly (e.g., amino acids of one portion may be directly joined to amino acids of a second portion via a peptide bond between terminal amino acids of the portions) or indirectly (e.g., amino acids of one portion of a fusion protein may be bound, for example by a first peptide bond, to a linker which is bound to a second portion of the fusion protein by a second peptide bond). In some embodiments, a first auxiliary protein is a first portion of a fusion protein and a second auxiliary protein is a second portion of the fusion protein. Connection of portions of fusion proteins using linkers is described further herein, for example in the section titled “Linkers”.

In some embodiments, a protein nanopore complex comprises multiple subunits, or monomers (e.g., multiple CsgG monomers), arranged around a central cavity or aperture (also referred to as a “lumen” of the nanopore). Formation of protein nanopores is described further herein, for example in the section titled “CsgG pores”. In some embodiments, one or more (e.g., 1, 2, 3, 4, 5, 6, 7, 8, 9, 10, 11, 12, 13, 14, 15, or more) auxiliary proteins are arranged in or with the lumen of the nanopore to form a continuous channel (e.g., a continuous lumen). In some embodiments, a protein nanopore complex comprises a ratio of pore monomers (e.g., CsgG pore monomers) to auxiliary proteins of 9:1, 9:2, 9:3, 9:4, 9:5, 9:6, 9:7, 9:8, 9:9 (e.g., 1:1), 9:10, 9:11, 9:12, 9:13, 9:14, 9:15, 9:16, 9:17, or 9:18 (e.g., 1:2) . . . . In some embodiments, the one or more auxiliary proteins or one or more fusion proteins may have the same symmetry as the nanopore.

For example, where the nanopore comprises eight monomers around a central axis, eight auxiliary proteins (or eight fusion proteins) are present, or where the nanopore comprises nine monomers around a central axis, nine auxiliary proteins (or nine fusion proteins) are present, etc. In some embodiments, the one or more auxiliary proteins (or one or more fusion proteins) may comprise more or fewer, such as one more or one fewer, monomers than the nanopore.

The lumen of a nanopore or a protein nanopore complex may have one or more constrictions. The “constriction”, “orifice”, “constriction region”, “channel constriction”, or “constriction site”, as used interchangeably herein, refers to an aperture defined by a luminal surface of a pore or protein pore complex, which acts to allow the passage of ions and target molecules (e.g., but not limited to polynucleotides or individual nucleotides) but not other non-target molecules through the pore or protein pore complex channel. The constriction(s) are typically the narrowest aperture(s) within a pore or protein pore complex or within the channel defined by the pore or pore complex. The constriction(s) may serve to limit the passage of molecules through the pore. The size of the constriction is typically a key factor in determining suitability of a pore or pore complex for analyte characterisation. If the constriction is too small, the molecule to be characterised will not be able to pass through. However, to achieve a maximal effect on ion flow through the channel, each constriction should not be too large. For example, each constriction should not be wider than the solvent-accessible transverse diameter of a target analyte. Ideally, each constriction should be as close as possible in diameter to the transverse diameter of the analyte passing through.

The number of constrictions in a protein pore complex described by the disclosure may vary. In some embodiments, a protein pore complex comprises at least 1, 2, 3, 4, 5, or more constrictions. In some embodiments, a protein pore complex comprises 2 or 3 constrictions. In some embodiments, a protein pore complex comprises 2 constrictions. In some embodiments, a first constriction is formed by a first auxiliary protein, and a second constriction is formed by a second auxiliary protein. In some embodiments, a first constriction formed by a portion of a CsgG nanopore, and a second constriction is formed by an auxiliary protein or a fusion protein. In some embodiments, a protein pore complex comprises 3 constrictions. In some embodiments, a first constriction is formed by a portion of a CsgG nanopore, a second constriction is formed by a first auxiliary protein, and a third constriction is formed by a second auxiliary protein. In some embodiments, a first constriction is formed by a portion of a CsgG nanopore and second and third constrictions are formed by a fusion protein.

The narrowest point of the central cavity or aperture typically forms a constriction in the continuous channel. In some embodiments, the diameter of a constriction is calculated by measuring the distance between the alpha-carbons (Cₐ) of the amino acid residues that extend furthest into the lumen of the nanopore to form the constriction. In some embodiments, the diameter of a constriction is calculated by measuring the distance between the Van der Waals radii of the atoms extending furthest into the lumen of the nanopore to form the constriction. In some embodiments, the minimum diameter of a constriction (e.g., a constriction formed by a portion of a CsgG protein, a constriction formed by an auxiliary protein, a constriction formed by a fusion protein, etc.) ranges from about 0.5 nm to about 4.0 nanometers (e.g., as measured by the distance between Van der Waals radii). In some embodiments, the minimum diameter of the constriction ranges from about 0.5 to about 3.0 nanometers or about 0.5 to about 2.0 nanometers, preferably from about 0.7 to about 1.8 nanometers, from about 0.8 to about 1.7 nanometers, from about 0.9 to about 1.6 nanometers, or from about 1.0 to about 1.5 nanometers, such as about 1.1, 1.2, 1.3 or 1.4 nanometers. In some embodiments, the minimum diameter of a constriction ranges from about 10 Å to about 30 Å, for example 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, or 30 Å (e.g., as measured by Cₐ to Cₐ). In some embodiments, the minimum diameter of a constriction ranges from about 10 Å to about 30 Å (e.g., as measured by Cₐ to Cₐ). In some embodiments, the minimum diameter of a constriction ranges from about 15 Å to about 25 Å (e.g., as measured by Cₐ to Cₐ).

The distance between the one or more constrictions in the lumen of a protein pore complex may vary. In some embodiments, the distance between the first constriction region and second constriction region ranges from about 5 Å to about 80 Å. In some embodiments, the distance between the first constriction region and the second constriction region is about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, or 80 Å in length. In some embodiments, the distance between the first constriction region and the second constriction region is more than 80 Å in length (e.g., 90 Å, 100 Å, etc.).

In some embodiments, the distance between a second constriction region and third constriction region ranges from about 5 Å to about 80 Å. In some embodiments, the distance between the first constriction region and the second constriction region is about 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, or 80 Å in length. In some embodiments, the distance between the second constriction region and the third constriction region is more than 80 Å in length (e.g., 90 Å, 100 Å, etc.).

In some embodiments, the distance between a first constriction region and third constriction region ranges from about 10 Å to about 160 Å. In some embodiments, the distance between the first constriction region and the second constriction region is about 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, 50 Å, 51 Å, 52 Å, 53 Å, 54 Å, 55 Å, 56 Å, 57 Å, 58 Å, 59 Å, 60 Å, 61 Å, 62 Å, 63 Å, 64 Å, 65 Å, 66 Å, 67 Å, 68 Å, 69 Å, 70 Å, 71 Å, 72 Å, 73 Å, 74 Å, 75 Å, 76 Å, 77 Å, 78 Å, 79 Å, 80 Å, 81 Å, 82 Å, 83 Å, 84 Å, 85 Å, 86 Å, 87 Å, 88 Å, 89 Å, 90 Å, 91 Å, 92 Å, 93 Å, 94 Å, 95 Å, 96 Å, 97 Å, 98 Å, 99 Å, 100 Å, 101 Å, 102 Å, 103 Å, 104 Å, 105 Å, 106 Å, 107 Å, 108 Å, 109 Å, 110 Å, 111 Å, 112 Å, 113 Å, 114 Å, 115 Å, 116 Å, 117 Å, 118 Å, 119 Å, 120 Å, 121 Å, 122 Å, 123 Å, 124 Å, 125 Å, 126 Å, 127 Å, 128 Å, 129 Å, 130 Å, 131 Å, 132 Å, 133 Å, 134 Å, 135 Å, 136 Å, 137 Å, 138 Å, 139 Å, 140 Å, 141 Å, 142 Å, 143 Å, 144 Å, 145 Å, 146 Å, 147 Å, 148 Å, 149 Å, 150 Å, 151 Å, 152 Å, 153 Å, 154 Å, 155 Å, 156 Å, 157 Å, 158 Å, 159 Å, or 160 Å in length. In some embodiments, the distance between the first constriction region and the third constriction region is more than 160 Å in length (e.g., 190 Å, 200 Å, etc.).

Auxiliary proteins (or fusion proteins) may, in some embodiments, be modified from their natural state to provide a constriction having a desired minimum diameter. For example, an auxiliary protein may be modified, such as by introducing one or more bulky residues by targeted mutation to create a constriction having a minimum diameter within the ranges specified above. The maximum height of an auxiliary protein is in one embodiment, from about 3 nm to about 20 nm, such as from about 4 nm to about 10 nm. In one embodiment, the length of the channel in the auxiliary protein is from about 3 nm to about 20 nm, such as from about 4 nm to about 10 nm. The height is the dimension of the auxiliary protein in a direction perpendicular to the membrane.

In some embodiments, an auxiliary protein (e.g., a first auxiliary protein or a second auxiliary protein) or a fusion protein (e.g., a first portion of a fusion protein or a second portion of a fusion protein) extends outside of the lumen of a protein pore complex. The auxiliary protein or fusion protein may extend outside the cis- or trans-side of the protein pore complex lumen (e.g., when the protein pore complex is inserted into a membrane). In some embodiments, the distance that an auxiliary protein or fusion protein extends outside of a lumen of a protein pore complex is calculated by measuring the distance of the Cₐ of the amino acid residue of the auxiliary protein or fusion protein that extends furthest outside of the lumen and a reference amino acid of the protein pore (e.g., a CsgG pore), for example amino acid residue Phe144 or Tyr196 of a wild-type CsgG monomer. In some embodiments, an auxiliary protein or fusion protein extends outside of the lumen by between about 0 Å and about 50 Å. In some embodiments, an auxiliary protein or fusion protein extends outside of the lumen by between about 5 Å and about 30 Å. In some embodiments, an auxiliary protein or fusion protein extends outside of the lumen by between about 10 Å and about 25 Å. In some embodiments, an auxiliary protein or fusion protein extends outside of the lumen by about 1 Å, 2 Å, 3 Å, 4 Å, 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, or about 50 Å.

The length between the first and second constrictions of a protein pore complex typically affects the axial length of the protein pore complex. In some embodiment, the axial length of a protein pore complex refers to the distance between the top of the lumen of the protein pore complex and the bottom of the lumen of the protein pore complex. In some embodiments, a protein pore complex has an axial length greater than 90 Å. In some embodiments, the axial length of a protein pore complex (e.g., a protein pore complex comprising one or more auxiliary proteins or one or more fusion proteins) ranges from about 95 Å to about 160 Å, for example 95 Å, 96 Å, 97 Å, 98 Å, 99 Å, 100 Å, 101 Å, 102 Å, 103 Å, 104 Å, 105 Å, 106 Å, 107 Å, 108 Å, 109 Å, 110 Å, 111 Å, 112 Å, 113 Å, 114 Å, 115 Å, 116 Å, 117 Å, 118 Å, 119 Å, 120 Å, 121 Å, 122 Å, 123 Å, 124 Å, 125 Å, 126 Å, 127 Å, 128 Å, 129 Å, 130 Å, 131 Å, 132 Å, 133 Å, 134 Å, 135 Å, 136 Å, 137 Å, 138 Å, 139 Å, 140 Å, 141 Å, 142 Å, 143 Å, 144 Å, 145 Å, 146 Å, 147 Å, 148 Å, 149 Å, 150 Å, 151 Å, 152 Å, 153 Å, 154 Å, 155 Å, 156 Å, 157 Å, 158 Å, 159 Å, or 160 Å.

In some embodiments, an auxiliary protein or fusion protein comprises one or more positively charged amino acids, such as arginine, lysine or histidine, or aromatic amino acids, such as tyrosine or tryptophan positioned at or close to (e.g. within about 1, 2, 3, 4 or 5 nm of the constriction), the constriction formed by the auxiliary protein or fusion protein. In some embodiments, an auxiliary protein or fusion protein comprises one or more polar amino acids, negative amino acids, or hydrophobic amino acids positioned at or close to (e.g. within about 1, 2, 3, 4 or 5 nm of the constriction), the constriction formed by the auxiliary protein or fusion protein. In some embodiments, the one or more amino acids positioned at or close to (e.g. within about 1, 2, 3, 4 or 5 nm of the constriction), the constriction formed by the auxiliary protein or fusion protein is asparagine, threonine, serine or glutamate. These amino acids typically facilitate the interaction between the pore and polynucleotides.

The positioning of the one or more auxiliary proteins (or one or more fusion proteins) of a protein pore complex may vary. In some embodiments, an auxiliary protein (or fusion protein) is positioned entirely within the lumen of a protein pore complex. In some embodiments, an auxiliary protein or a fusion protein comprises a portion that extends beyond the lumen of the protein pore complex, for example extending above the lumen of the protein pore complex (e.g., extending above the cap region on the cis-side of the protein pore complex) and/or extending below the protein pore complex (e.g., extending below the transmembrane domain (e.g., barrel) on the trans-side of the protein pore complex). In some embodiments, an auxiliary protein or a fusion protein (or a portion of an auxiliary protein or fusion protein, such as a first portion or second portion) is attached to a nanopore (e.g., a CsgG nanopore). In some embodiments, the auxiliary protein or fusion protein (or portion thereof) is attached to the nanopore covalently. In some embodiments, the auxiliary protein or fusion protein (or portion thereof) is attached to the nanopore non-covalently. In some embodiments, a first auxiliary protein and a second auxiliary protein are attached to one another (e.g., covalently attached, non-covalently attached, etc.). In some embodiments, a first portion of a fusion protein and a second portion of a fusion protein are attached to one another (e.g., covalently attached, non-covalently attached, etc.). In some embodiments, an auxiliary protein or a fusion protein (or a portion of an auxiliary protein or fusion protein, such as a first portion or second portion) is not attached to a nanopore (e.g., a CsgG nanopore). In some embodiments, a first auxiliary protein and a second auxiliary protein are not attached to one another.

In some embodiments, an auxiliary protein (e.g., a first auxiliary protein) is not CsgF or a CsgF peptide or a functional homologue, fragment or modified version thereof. In some embodiments, portion of a fusion protein (e.g., a first portion and/or a second portion) is not CsgF or a CsgF peptide or a functional homologue, fragment or modified version thereof. In some embodiments, an auxiliary protein is not a CsgG nanopore, or a homologue, fragment or modified version thereof. In some embodiments, a portion of a fusion protein (e.g., a first portion and/or a second portion) is not a CsgG nanopore, or a homologue, fragment or modified version thereof.

In some embodiments, an auxiliary protein is not a polynucleotide binding protein. In some embodiments, an auxiliary protein is not a functional polynucleotide binding protein, e.g. an auxiliary protein is not a polynucleotide binding protein having enzymatic activity. some embodiments, an auxiliary protein may be a protein other than a nucleic acid handling enzyme, for example, an auxiliary protein that is not a helicase or a polymerase, or a protein derived from such an enzyme. In some embodiments, an auxiliary protein has no enzymatic activity. In some embodiments, an auxiliary protein does not undergo a conformational change upon passage of a target analyte through the continuous channel formed in the protein pore complex.

In some embodiments, an auxiliary protein or fusion protein (e.g., portion of a fusion protein) is a component of a nanopore system, or a modified component of such a system, other than a component that forms a transmembrane pore. An example of such a component is CsgF, or a truncated version of CsgF. In some embodiments, an auxiliary protein or fusion protein comprises a CsgF protein, or a homologue or modified version, such as a fragment, thereof. In some embodiments, the pore complex comprises a CsgF protein or peptide and a non-CsgG pore, homologue or modified version, such as a fragment, thereof.

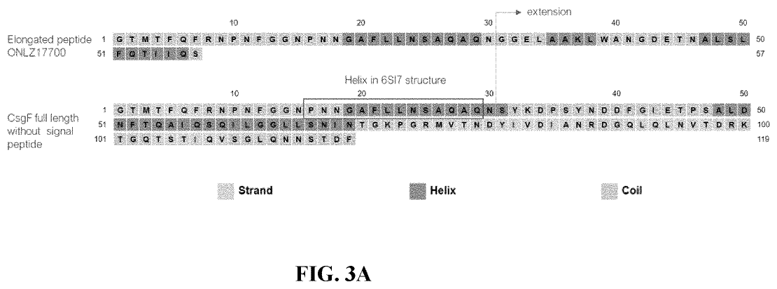

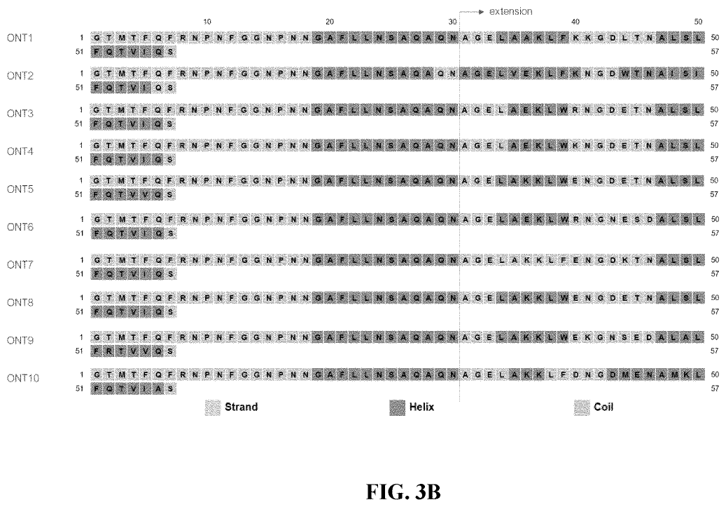

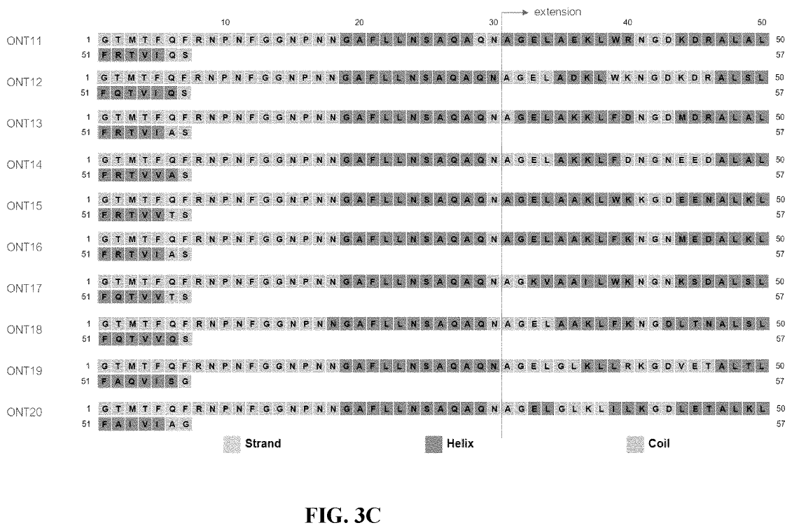

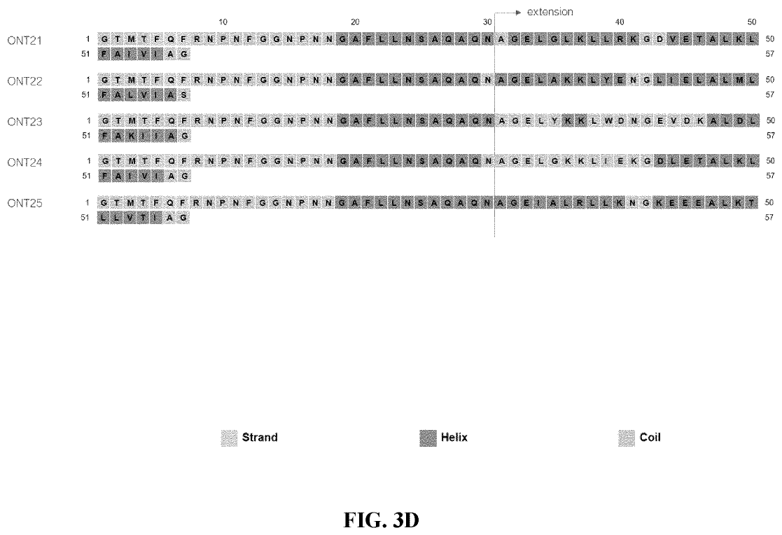

The term “CsgF protein” or “CsgF peptide” preferably defines a CsgF peptide that has been truncated from its C-terminal end (i.e., is an N-terminal fragment). The CsgF peptide may be a fragment of wild-type E. coli CsgF (e.g., as shown in FIG. 3A), or of a wild-type homologue of E. coli CsgF, such as for example, a peptide comprising any one of the amino acid sequences shown in WO 2019/002893 (incorporated by reference herein in its entirety). A CsgF homologue is referred to as a polypeptide that has at least 30%, 40%, 50%, 60%, 70%, 80%, 90%, 95% or 99% complete sequence identity to wild-type E. coli CsgF. A CsgF homologue may also be referred to as a polypeptide that contains the PFAM domain PF10614, which is characteristic for CsgF-like proteins. A list of presently known CsgF homologues and CsgF architectures can be found at http://pfam.xfam.org//family/PF10614. Mature CsgF (e.g., as shown in FIG. 3A) can be divided into three main regions: a “CsgF constriction peptide” (FCP), a “neck” region and a “head” region. The “head” region of the CsgF peptide is distinct from a constriction of a pore as described herein. The “head” region of the CsgF peptide may also be referred to as the “C-terminal head domain”. The structure of CsgF is discussed in detail in WO 2019/002893 (incorporated by reference herein in its entirety).

In some embodiments, a CsgF peptide is a truncated CsgF peptide lacking the C-terminal head; lacking the C-terminal head and a part of the neck domain of CsgF (e.g., the truncated CsgF peptide may comprise only a portion of the neck domain of CsgF); or lacking the C-terminal head and neck domains of CsgF. The CsgF peptide may lack part of the CsgF neck domain, e.g., the CsgF peptide may comprise a portion of the neck domain, such as for example, from amino acid residue 36 at the N-terminal end of the neck domain (e.g., residues 36-40, 36-41, 36-42, 36-43, 36-45,36-46 up to residues 36-50 or 36-60 of wild-type E. coli CsgF). In some embodiments, a CsgF peptide comprises a CsgG-binding region and a region that forms a constriction in the lumen of pore. The CsgG-binding region typically comprises residues 1 to 11 and/or 29 to 32 of the CsgF protein (e.g., wild-type E. coli CsgF or a homologue from another species) and may include one or more modifications. The region that forms a constriction in the pore typically comprises residues 9 to 28 of the CsgF protein (e.g., wild-type E. coli CsgF or a homologue from another species) and may include one or more modifications. In some embodiments, residues 9 to 17 comprise a conserved motif, N₉PXFGGXXX₁₇, and form a turn region. In some embodiments, residues 9 to 28 form an alpha-helix. In some embodiments, the amino acid residue at position 17 of a CsgF peptide forms the apex of the constriction region, corresponding to the narrowest part of the CsgF constriction in the pore. In some embodiments, a CsgF constriction region also makes stabilising contacts with a CsgG beta-barrel, primarily at residues 8, 9, 11, 12, 18, 21 and 22 of the CsgF peptide. In some embodiments, a CsgF peptide comprises or consists of the amino acid sequence

GTMTFQFRNPNFGGNPNNGAFLLNSAQAQN (SEQ ID NO: 60), which corresponds to amino acid residues 1-30 of wild-type E. coli CsgF. In some embodiments, a CsgF peptide is a first auxiliary protein. In some embodiments, a CsgF peptide is a portion (e.g., a first portion or a second portion) of a fusion protein. In some embodiments, a CsgF peptide comprises or consists of amino acid residues 1-23 of wild-type E. coli CsgF. In some embodiments, a CsgF peptide comprises or consists of amino acid residues 1-23 of wild-type E. coli CsgF. In some embodiments, a CsgF peptide comprises or consists of amino acid residues 1-24 of wild-type E. coli CsgF. In some embodiments, a CsgF peptide comprises or consists of amino acid residues 1-24 of wild-type E. coli CsgF.

In some embodiments, a CsgF peptide has a length of from 28 to 60 amino acids, such as 29 to 49, 30 to 45 or 32 to 40 amino acids. In some embodiments, a CsgF peptide comprises from 29 to 35 amino acids, or 29 to 45 amino acids. In some embodiments, a CsgF peptide comprises a length of 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a CsgF peptide comprises all or part of the FCP, which corresponds to residues 1 to 35 of wild-type E. coli CsgF (or corresponding residues in CsgF homologs). In some embodiments, where the CsgF peptide is shorter that the FCP, the truncation is preferably made at the C-terminal end.

In the CsgF peptide, one or more residues may be modified. For example, the CsgF peptide may comprise a modification at a position corresponding to one or more of the following positions in SEQ ID NO: 6: G1, M3, T4, F5, R8, N9, N11, F12, N17, A20, N24, A26 and Q29 of SEQ ID NO: 60. In some embodiments, a CsgF peptide is modified to introduce one or more cysteines, one or more hydrophobic amino acids, one or more charged amino acids, one or more non-native amino acids, one or more polar amino acids, or one or more photoreactive amino acids, for example at a position corresponding to one or more of the following positions in SEQ ID NO: 60: G1, T4, F5, R8, N9, N11, F12, N17, A20, N24, A26, Q27, and Q29. Any number and combination of such introductions may be made. The introduction is preferably by substitution.

In some embodiments, a CsgF peptide comprises a modification at a position corresponding to one or more of the following positions in SEQ ID NO: 60: N15, N17, A20, N24 and A28. In some embodiments, a CsgF peptide comprises one or more of the substitutions:

N15S/A/T/Q/G/L/V/I/F/Y/W/R/K/D/C/E;
N17S/A/T/Q/G/L/V/I/F/Y/W/R/K/D/C/E;
A20S/T/Q/N/G/L/V/I/F/Y/W/R/K/D/C/E;
N24S/T/Q/A/G/L/V/I/F/Y/W/R/K/D/C/E;
or
A28S/T/Q/N/G/L/V/I/F/Y/W/R/K/D/C/E.

In some embodiments a CsgF peptide is preferably a variant of any of the CsgF sequences discussed above, including SEQ ID NO: 60, comprising one or more modifications compared with the comparative sequence. Over the entire length of the amino acid sequence of SEQ ID NO: 60, a variant will preferably be at least 40% homologous to that sequence based on amino acid identity. More preferably, the variant may be at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 60 over the entire sequence. Over the entire length of the amino acid sequence of SEQ ID NO: 60, a variant will preferably be at least 40% identical to that sequence. More preferably, the variant may be at least 45%, at least 50%, at least 55%, at least 60%, at least 65%, at least 70%, at least 75%, at least 80%, at least 85%, at least 90% and more preferably at least 95%, 97% or 99% identical to SEQ ID NO: 60 over the entire sequence. There may be at least 80%, for example at least 85%, 90% or 95%, amino acid identity over a stretch of 15 or more, for example 15, 16, 17, 18, 19, 20, 21, 22, 23, 24, 25, 26, 27, 28, 29, 30, or more, contiguous amino acids (“hard homology”). These levels of homology/identity equally apply to any of the other CsgF peptides described above.

Any number of the CsgF peptides in the pore or pore complex, such as 1, 2, 3, 4, 5, 6, 7, 8, 9 or 10, may contain one or more substitutions compared with SEQ ID NO: 60. In some embodiments, all six to ten monomers in a pore or pore complex preferably contain one or more substitutions compared with SEQ ID NO: 60. The CsgF peptides in the pore complex may be the same or different. The CsgF peptides are preferably identical in each pore monomer conjugate in the pore complex of the disclosure.

Aspects of the disclosure relate to auxiliary proteins or fusion proteins that comprise one or more alpha helices. In some embodiments, such proteins may be referred to as “helix-forming proteins”. The disclosure is based, in part, on the recognition that helix-forming proteins may be positioned in the lumen of certain nanopores (e.g., CsgG nanopores) to form one or more constrictions in the lumen of the nanopore, and that the presence of such one or more constrictions improves the signal to noise ratio (e.g., discrimination of polynucleotide bases) of the resulting protein pore complex. The term “helix” or “helical” generally refers to a coiled structural arrangement of a protein that forms a spiral and results from formation of hydrogen bonds between backbones of non-contiguous amino acid residues in a repeating pattern. In some embodiments, a helix is an alpha-helix (also referred to as a 3.613-helix), which comprises about 3.6 amino acid residues per helical turn, with 13 atoms being involved in the ring formed by the hydrogen bonds. In some embodiments, a helix is a 3₁₀ helix, which comprises a about three residues per turn, and has 10 atoms in the ring formed by making the hydrogen bond.

The number of helices (e.g., alpha helices, 3₁₀ helices, π helices, etc.) in an auxiliary protein or a fusion protein may vary. In some embodiments, the number of helices in an auxiliary protein (e.g., a first auxiliary protein, second auxiliary protein, etc.) ranges from about 0 to about 15, for example 0, 1, 2, 3, 4, 5, 6, 7, 8, 9, 10, 11, 12, 13, 14, or 15. In some embodiments, the number of helices in an auxiliary protein (e.g., a first auxiliary protein, second auxiliary protein, etc.) is more than 15 (e.g., 20, 25, etc.). In some embodiments, a fusion protein (e.g., a first portion of a fusion protein, second portion of a fusion protein, etc.) comprises between 0 to about 15 helices (e.g., alpha helices, 3₁₀ helices, π helices, etc.), for example 0, 1, 2, 3, 4, 5, 6, 7, 8, 9, 10, 11, 12, 13, 14, or 15 helices.

The number of turns in a helix (e.g., an alpha helix, 3₁₀ helix, π helix, etc.) may vary. In some embodiments, each helix (e.g., alpha helix, 3₁₀ helix, π helix, etc.) of an auxiliary protein or fusion protein comprises from about 0 to about 15 helical turns, for example 0, 1, 2, 3, 4, 5, 6, 7, 8, 9, 10, 11, 12, 13, 14, or 15 helical turns. A helix (e.g., an alpha helix, 3₁₀ helix, π helix, etc.) may comprise 1 or more half-helices (e.g., half-turns), for example, 0.5, 1.5, 2.5, 3.5 4.5, 5.5, 6.5, 7.5, 8.5, 9.5, 10.5, 11.5, 12.5, 13.5, 14.5, etc. helical turns.

The number of amino acids forming a helix (e.g., alpha helix, 3₁₀ helix, π helix, etc.) may vary. In some embodiments, each helix (e.g., alpha helix, 3₁₀ helix, π helix, etc.) of an auxiliary protein or fusion protein comprises between 2 and 55 amino acid residues, for example 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acid residues.

The angles of the helices of an auxiliary protein or fusion protein may vary. In some embodiments a helix comprises Phi angles ranging from about −45° to −90° (e.g., −45°, −46°, −47°, −48°, −49°, −50°, −51°, −52°, −53°, −54°, −55°, −56°, −57°, −58°, −59°, −60°, −61°, −62°, −63°, −64°, −65°, −66°, −67°, −68°, −69°, −70°, −71°, −72°, −73°, −74°, −75°, −76°, −77°, −78°, −79°, −80°, −81°, −82°, −83°, −84°, −85°, −86°, −87°, −88°, −89°, or)−90°. In some embodiments a helix comprises Psi angles ranging from about 0° to −70° (e.g., 0°, −1°, −2°, −3°, −4°, −5°, −6°, −7°, −8°, −9°, −10°, −11°, −12°, −13°, −14°, −15°, −16°, −17°, −18°, −19°, −20°, −21°, −22°, −23°, −24°, −25°, −26°, −27°, −28°, −29°, −30°, −31°, −32°, −33°, −34°, −35°, −36°, −37°, −38°, −39°, −40°, −41°, −42°, −43°, −44°, −45°, −46°, −47°, −48°, −49°, −50°, −51°, −52°, −53°, −54°, −55°, −56°, −57°, −58°, −59°, −60°, −61°, −62°, −63°, −64°, −65°, −66°, −67°, −68°, −69°, or −70°). In some embodiments, each of the helices comprises 1-20 amino acid residues having Phi angles ranging from about −45° to −90° and Psi angles ranging from about 0° to −70°. In some embodiments, each of the helices comprises 1-30 amino acid residues having Phi angles ranging from about −45° to −90° and Psi angles ranging from about 0° to −70°.

In some embodiments, one or more helices of auxiliary proteins or a fusion protein comprise structural features which facilitate packing of the helices together. “Packing” of helices typically refers to the tight association of two or more helices with one another due to covalent or non-covalent interactions between the helices, for example salt bridges, hydrogen bonds, disulfide bonds and tight hydrophobic side chain to side chain contacts, side chain to main chain contacts, main chain to main chain contacts, etc., as described by Walther and Argos, J Mol Biol. 1996 Jan. 26; 255 (3): 536-53. doi: 10.1006/jmbi.1996.0044. Methods of predicting helical packing are known, for example as described by Eilers et al. Proc Natl Acad Sci USA. 2000 May 23; 97 (11): 5796-5801.

Aspects of the disclosure relate to the recognition that fusion proteins that are cyclized improve discrimination of target analytes in protein pore complexes. A “cyclised” protein typically refers to a protein (e.g., a fusion protein) which comprises one or more intramolecular interactions that result in formation of one or more circular arrangements of bonds. Examples of cyclisation include side chain-to-side chain cyclisation (e.g., intramolecular disulfide bond formation), head-to-tail cyclisation (e.g., formation of amide bonds between the N- and C-terminal amino acids of a protein), tail-to-side chain cyclisation, and head-to-side chain cyclisation, for example as described in Hayes et al. Org Biomol Chem. 2021 May 12; 19 (18): 3983-4001. In some embodiments, a fusion protein comprises one or more side-chain to side-chain cyclisation bonds. In some embodiments, at least one of the side-chain to side-chain cyclisation bonds is a disulfide bond. In some embodiments, the one or more cyclisation bonds results in cyclisation between the first portion of the fusion protein and the second portion of the fusion protein (e.g., cyclisation between a CsgF peptide and a helix-forming protein). In some embodiments, an auxiliary protein or a fusion protein comprises a loop region (e.g., a linker forming a loop region) comprising one or more cyclisation bonds. In some embodiments, the cyclisation bond is formed by a chemical crosslinker and/or comprises a disulfide bond.

### CsgG Nanopores

Aspects of the disclosure relate to protein pore complexes. In some embodiments, protein pore complexes described by the disclosure comprise a nanopore (e.g., a CsgG nanopore). A nanopore is a hole or channel through a membrane that permits hydrated ions driven by an applied potential to flow across or within the membrane.

The nanopore is, in some embodiments, a transmembrane protein pore. The transmembrane protein pore typically spans the entire membrane and may have a structure that extends beyond the membrane on one or both sides. A transmembrane protein pore is a single or multimeric protein that permits hydrated ions to flow from one side of a membrane to the other side of the membrane. The transmembrane protein pore comprises a channel that allows an analyte, for example a polynucleotide, such as DNA or RNA, to move, or be moved, into and/or through the pore.

The transmembrane protein pore typically comprises a barrel or channel through which the ions may flow. The subunits of the pore typically surround a central axis and contribute strands to a transmembrane β-barrel or channel or a transmembrane α-helix bundle or channel. The barrel or channel of the transmembrane protein pore typically comprises amino acids that facilitate interaction with polynucleotides. These amino acids are preferably located near a constriction (such as within 1, 2, 3, 4 or 5 nm) of the barrel or channel. The transmembrane protein pore typically comprises one or more polar or hydrophobic residues. These amino acids typically facilitate the interaction between the pore and nucleotides, polynucleotides or nucleic acids.

In some embodiments, the nanopore is a CsgG pore, such as for example CsgG from E. coli Str. K-12 substr. MC4100, or a homologue or mutant thereof. Mutant CsgG pores may comprise one or more mutant monomers. The CsgG pore may be a homopolymer comprising identical monomers, or a heteropolymer comprising two or more different monomers. Suitable pores derived from CsgG are disclosed in WO 2016/034591, WO2017/149316, WO2017/149317, WO2017/149318, International patent Application Numbers PCT/GB2018/051191 and PCT/GB2018/051858, and Chinese patent publication numbers CN113773373, CN113896776, CN113912683, and CN113754743, each of which is incorporated by reference herein in its entirety. Additional examples of CsgG pores include but are not limited to Uniprot reference numbers K4KIX7, A0A086DIN6, A0A1I1MNE8, A0A143HJG2, AoA090RS48, and A0A090SZM0.

CsgG pores typically comprise one or more CsgG monomers. A CsgG pore monomer is a monomer that is capable of forming a CsgG pore. Such monomers are known in the art, especially from WO 2019/002893 (incorporated by reference herein in its entirety). The CsgG pore preferably comprises one or more of (a) a cap region, (b) a constriction region, and (c) a transmembrane beta barrel region, such as (a), (b), (c), (a) and (b), (a) and (c), (b) and (c), or (a), (b) and (c). The CsgG pore monomer preferably comprises one or more of (a) a cap forming region, (b) a constriction forming region, and (c) a transmembrane beta barrel forming region, such as (a), (b), (c), (a) and (b), (a) and (c), (b) and (c), or (a), (b) and (c). The CsgG pore formed by the monomer may have any structure but preferably has or comprises the structure of a wild-type E. coli CsgG pore (e.g., as described by PDB Accession Number 4UV3). The protein structure of CsgG defines a channel or hole that allows the translocation of molecules and ions from one side of the membrane to the other.

The CsgG pore may be any size but preferably has the dimensions of the wild-type E. coli CsgG pore (e.g., as described by PDB Accession Number 4UV3). These dimensions are shown in FIG. 19. In some embodiments, the CsgG pore has an external diameter of from about 100 to about 150 Å at its widest point, such as from about 110 to about 140 Å or from about 115 to about 125 Å at its widest point. In some embodiments, the CsgG pore has an external diameter of about 120 Å at its widest point. In some embodiments, the CsgG pore has a total length of from about 80 to about 120 Å, such as from about 90 to about 110 Å or from about 95 to about 105 Å. In some embodiments, the CsgG pore has a total length of about 98 Å. References to “total length” and “length” relate to the length of the pore or pore region when viewed from the side (see, e.g., as a cis-to-trans cross-section of the pore inserted into a membrane). This may be the side view in FIG. 19. In some embodiments, the external diameter is measured by calculating the Cₐ to Cₐ distance of the amino acid residues on the exterior of the CsgG pore that are furthest apart. In some embodiments, the external diameter is measured by calculating the distance of Van der Waals radii of the amino acid residues on the exterior of the CsgG pore that are furthest apart.

In some embodiments, the cap region has a length of from about 20 to about 60 Å, such as from about 30 to about 50 Å or from about 35 to about 45 Å. In some embodiments, the cap region has a length of about 39 Å. In some embodiments, the channel defined by the cap region has an opening of from about 30 to about 70 Å in diameter, such as from about 40 to about 60 Å or from about 45 to about 55 Å in diameter. In some embodiments, the channel defined by the cap region has an opening of about 66 Å in diameter. In some embodiments, the channel defined by the cap region is from about 20 to about 66 Å in diameter at its narrowest point, such as from about 30 to about 50 Å or from about 32 to about 43 Å in diameter at its narrowest point. In some embodiments, the channel defined by the cap region is preferably about 43 Å in diameter at its narrowest point. In some embodiments, the external diameter is measured by calculating the Cₐ to Cₐ distance of the amino acid residues on the channel of the cap region of the CsgG pore that are closest together. In some embodiments, the external diameter is measured by calculating the distance of Van der Waals radii of the amino acid residues on the channel of the cap region that are closest together.

In some embodiments, the constriction region formed by the CsgG pore (when present) has a length of from about 5 to about 40 Å, such as from about 10 to about 30 Å or from about 15 to about 25 Å. In some embodiments, the constriction region has a length of about 20 Å. In some embodiments, the channel defined by the constriction region is from about 2 to about 30 Å in diameter at its narrowest point, such as from about 5 to about 25 Å, from about 8 to about 20 Å or from about 10 to about 15 Å in diameter at its narrowest point. In some embodiments, the channel defined by the constriction region is about 9 Å in diameter. In some embodiments, the channel defined by the constriction region is about 18.5 Å in diameter. In some embodiments, the constriction is from about 2 to about 30 Å in diameter, such as from about 5 to about 25 Å, from about 8 to about 20 Å or from about 10 to about 15 Å in diameter. In some embodiments, the constriction is about 12 Å in diameter. In some embodiments, the constriction region of a CsgG pore is measured by calculating the Cₐ to Cₐ distance of the amino acid residues that extend furthest into the lumen of the pore and form the constriction. In some embodiments, the external diameter is measured by calculating the distance of Van der Waals radii of the amino acid residues that extend furthest into the lumen of the pore and form the constriction.

In some embodiments, the transmembrane beta barrel region has a length of from about 20 to about 60 Å, such as from about 30 to about 50 Å or from about 35 to about 45 Å. In some embodiments, the transmembrane beta barrel has a length of about 39 Å. In some embodiments, the channel defined by the transmembrane beta barrel region is from about 20 to about 60 Å in diameter at its narrowest point, such as from about 30 to about 50 Å or from about 35 to about 45 Å in diameter at its narrowest point. In some embodiments, the channel defined by the transmembrane beta barrel region is about 55 Å in diameter at its narrowest point.

All of the measurements above are based on measuring from backbone to backbone of the amino acids forming the different regions (as shown in FIG. 19).

SEQ ID NO: 59 shows the sequence of wild-type E. coli CsgG as a mature protein. Residues 1 to 41 of SEQ ID NO: 59 form the cap region. Residues 64 to 131 of SEQ ID NO: 59 form the constriction region. Residues 156 to 180 and 212 to 262 of SEQ ID NO: 59 form the transmembrane beta barrel region.

In some embodiments, the CsgG pore monomer is a variant of SEQ ID NO: 59 because it has a cysteine at a position corresponding to position 153 or 133 of SEQ ID NO: 59. In some embodiments, the variant CsgG monomer may also be referred to as a modified CsgG pore monomer or a mutant CsgG pore monomer. The modifications, or mutations, in the variant include but are not limited to any one or more of the modifications disclosed herein, or combinations of said modifications. The CsgG pore monomer may be a CsgG homologue monomer. A CsgG homologue monomer is a polypeptide that has at least 40%, 50%, 60%, 70%, 80%, 85%, 90%, 95% or 99% complete sequence identity to wild-type E. coli CsgG as shown in SEQ ID NO: 59. A CsgG homologue is also referred to as a polypeptide that contains the PFAM domain PF03783, which is characteristic for CsgG-like proteins. A list of presently known CsgG homologues and CsgG architectures can be found at http://pfam.xfam.org//family/PF03783.

In some embodiments, the CsgG pore monomer is a variant of SEQ ID NO: 59 comprising one or more modifications in addition to the cysteine at a position corresponding to position 153 or 133 in SEQ ID NO: 59. Over the entire length of the amino acid sequence of SEQ ID NO: 59, a variant will preferably be at least 40% homologous to that sequence based on amino acid identity. More preferably, the variant may be at least 45%, at least 50%, at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 59 over the entire sequence. Over the entire length of the amino acid sequence of SEQ ID NO: 59, a variant will preferably be at least 40% identical to that sequence. More preferably, the variant may be at least 45%, at least 50%, at least 55%, at least 60%, at least 65%, at least 70%, at least 75%, at least 80%, at least 85%, at least 90% and more preferably at least 95%, 97% or 99% identical to SEQ ID NO: 59 over the entire sequence.

Sequence identity can also relate to a fragment or portion of the CsgG pore monomer. Hence, a sequence may have less than 40% overall sequence homology/identity with SEQ ID NO: 59, but the sequence of a particular region, domain or subunit could share at least 80%, 90%, or as much as 99% sequence homology/identity with the corresponding region of SEQ ID NO: 59. There may be at least 80%, for example at least 85%, 90% or 95%, amino acid identity over a stretch of 100 or more, for example 125, 150, 175 or 200 or more, contiguous amino acids (“hard homology”). In some embodiments, the CsgG pore monomer is preferably a variant of SEQ ID NO: 3 comprising a sequence that is at least 40% homologous to the cap region of SEQ ID NO: 3 (residues 1 to 41). More preferably, the variant may comprise a sequence that is at least 45%, at least 50%, at least 55%, at least 60%, at least 65%, at least 70%, at least 75%, at least 80%, at least 85%, at least 90% and more preferably at least 95%, 97% or 99% homologous based on amino acid identity to residues 1 to 41 of SEQ ID NO: 59. In some embodiments, the variant comprises a sequence that is at least 40% identical to residues 1 to 41 of SEQ ID NO: 59. In some embodiments, the variant comprises a sequence that is at least 45%, at least 50%, at least 55%, at least 60%, at least 65%, at least 70%, at least 75%, at least 80%, at least 85%, at least 90% and more preferably at least 95%, 97% or 99% identical to residues of 1 to 41 of SEQ ID NO: 59.

In some embodiments, the CsgG pore monomer is a variant of SEQ ID NO: 59 comprising a sequence that is at least 40% homologous to the constriction region of SEQ ID NO: 59 (residues 64 to 131). In some embodiments, the variant comprises a sequence that is at least 45%, at least 50%, at least 55%, at least 60%, at least 65%, at least 70%, at least 75%, at least 80%, at least 85%, at least 90% and more preferably at least 95%, 97% or 99% homologous based on amino acid identity to residues 64 to 131 of SEQ ID NO: 59. In some embodiments, the variant comprises a sequence that is at least 40% identical to residues 64 to 131 of SEQ ID NO: 59. In some embodiments, the variant comprises a sequence that is at least 45%, at least 50%, at least 55%, at least 60%, at least 65%, at least 70%, at least 75%, at least 80%, at least 85%, at least 90% and more preferably at least 95%, 97% or 99% identical to residues 64 to 131 of SEQ ID NO: 59.

In some embodiments, the CsgG pore monomer is a variant of SEQ ID NO: 59 comprising a sequence that is at least 40% homologous to the transmembrane beta barrel region of SEQ ID NO: 3 (residues 156-180 and 212-262). In some embodiments, the variant comprises a sequence that is at least 45%, at least 50%, at least 55%, at least 60%, at least 65%, at least 70%, at least 75%, at least 80%, at least 85%, at least 90% and more preferably at least 95%, 97% or 99% homologous based on amino acid identity to residues 156-180 and 212-262 of SEQ ID NO: 59. In some embodiments, the variant comprises a sequence that is at least 40% identical to residues 156-180 and 212-262 of SEQ ID NO: 59. In some embodiments, the variant comprises a sequence that is at least 45%, at least 50%, at least 55%, at least 60%, at least 65%, at least 70%, at least 75%, at least 80%, at least 85%, at least 90% and more preferably at least 95%, 97% or 99% identical to residues 156-180 and 212-262 of SEQ ID NO: 59.

CsgG pore monomers are highly conserved (as can be readily appreciated from FIGS. 45 to 47 of WO 2017/149317). Furthermore, from knowledge of the mutations in relation to SEQ ID NO: 59 it is possible to determine the equivalent positions for mutations of CsgG pore monomers other than that of SEQ ID NO: 59.

Thus, reference to a mutant CsgG pore monomer comprising a variant of the sequence as shown in SEQ ID NO: 59 and specific amino-acid mutations thereof as set out in the claims and elsewhere in the specification also encompasses a mutant CsgG pore monomer comprising a variant of any of the sequences shown in SEQ ID NOs: 68 to 88 of WO 2019/002893 (incorporated by reference herein in its entirety) and corresponding amino-acid mutations thereof. The CsgG pore monomer may also be any of the sequences shown in CN 113773373 A, CN 113896776 A, CN 113912683 A, and CN 113754743 A or a variant thereof.

Standard methods in the art may be used to determine homology. For example, the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (http://www.ncbi.nlm.nih.gov/).

SEQ ID NO: 59 is the wild-type CsgG pore monomer from Escherichia coli Str. K-12 substr. MC4100. A variant of SEQ ID NO: 59 may comprise any of the substitutions present in another CsgG homologue. Preferred CsgG homologues are shown in SEQ ID NOs: 68 to 88 of WO 2019/002893 (incorporated by reference herein in its entirety). The variant may comprise combinations of one or more of the substitutions present in SEQ ID NOs: 68 to 88 WO 2019/002893 (incorporated by reference herein in its entirety) compared with SEQ ID NO: 59, including one or more

The CsgG pore monomer in the pore monomer conjugate of the disclosure typically retains the ability to form the same 3D structure as the wild-type CsgG pore monomer, such as the same 3D structure as a CsgG pore monomer having the sequence of SEQ ID NO: 59. The 3D structure of CsgG is known in the art and is disclosed, for example, in Goyal et al (2014) Nature 516 (7530): 250-3. Any number of mutations may be made in the wild-type CsgG sequence in addition to the mutations described herein provided that the CsgG pore monomer retains the improved properties imparted on it by the mutations.

Amino acid substitutions may be made to the amino acid sequence of SEQ ID NO: 59 in addition to those discussed above, for example up to 1, 2, 3, 4, 5, 10, 20 or 30 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid.

In some embodiments, the CsgG pore monomer is modified to introduce one or more cysteines, one or more hydrophobic amino acids, one or more charged amino acids, one or more non-native amino acids, one or more polar amino acids, or one or more photoreactive amino acids. Any number and combination of such introductions may be made. The introduction is preferably by substitution.

One or more amino acid residues of the amino acid sequence of SEQ ID NO: 59 may additionally be deleted from the polypeptides described above. Up to 1, 2, 3, 4, 5, 10, 20 or 30 or more residues may be deleted.

Variants may include fragments of SEQ ID NO: 59. Such fragments retain pore forming activity. Fragments may be at least 50, at least 100, at least 150, at least 200 or at least 250 amino acids in length. Such fragments may be used to produce the pores. A fragment preferably comprises the membrane spanning domain of SEQ ID NO: 59, namely K135-Q153 and S183-S208.

One or more amino acids may be alternatively or additionally added to the polypeptides described above. An extension may be provided at the amino terminal or carboxy terminal of the amino acid sequence of SEQ ID NO: 59 or polypeptide variant or fragment thereof. The extension may be quite short, for example from 1 to 10 amino acids in length. Alternatively, the extension may be longer, for example up to 50 or 100 amino acids. A carrier protein may be fused to an amino acid sequence. Other fusion proteins are discussed in more detail elsewhere in the disclosure, for example in the section titled “Auxiliary Proteins”.

A variant of SEQ ID NO: 59 is a polypeptide that has an amino acid sequence which varies from that of SEQ ID NO: 59 and which retains its ability to form a pore. A variant typically contains the regions of SEQ ID NO: 59 that are responsible for pore formation. The pore forming ability of CsgG, which contains a β-barrel, is provided by β-sheets in the transmembrane beta barrel region of each subunit monomer. A variant of SEQ ID NO: 59 typically comprises the regions in SEQ ID NO: 59 that form β-sheets, namely K134-Q154 and S183-S208. One or more modifications can be made to the regions of SEQ ID NO: 3 that form β-sheets as long as the resulting variant retains its ability to form a pore.

The one or more modifications in the CsgG pore monomer preferably improve the ability of a pore complex comprising the pore monomer to characterise an analyte. For example, modifications/mutations/substitutions are contemplated to alter the number, size, shape, placement or orientation of the constriction within a channel from the pore monomer conjugate of the disclosure. The CsgG pore monomer or the variant of SEQ ID NO: 59 may have any of the particular modifications or substitutions disclosed in WO 2016/034591, WO 2017/149316, WO 2017/149317, WO 2017/149318, WO 2018/211241, and WO 2019/002893 (all incorporated by reference herein in their entirety).

Preferred modifications or substitutions in SEQ ID NO: 59 include, but are not limited to, one or more of, such as 2 or more, 3 or more, 4 or more, 5 or more, 6 or more, 7 or more or all of:
(a) a substitution at position Y51, such as Y51I, Y51L, Y51A, Y51V, Y51T, Y51S, Y51Q or Y51N;
(b) a substitution at position N55, such as N55I, N55L, N55A, N55V, N55T, N55S or N55Q;
(c) a substitution at position F56, such as F56I, F56L, F56A, F56V, F56T, F56S, F56Q or F56N;
(d) a substitution at position L90, such as L90N, L90D, L90E, L90R or L90K;
(e) a substitution at position N91, such as N91D, N91E, N91R or N91K;
(f) a substitution at position K94, such as K94R, K94F, K94Y, K94Q, K94W, K94L, K94S or K94N;
(g) a substitution at position R192, such as R192Q, R192F, R192S R192D, or R192T; and
(i) a substitution at position C215, such as C215T, C215S, C215I, C215L, C215A, C215V, or C215G.

The variant of SEQ ID NO: 3 may further comprise a deletion of one or more positions, such as a deletion of T104-N109, a deletion of F193-L199 or a deletion of F195-L199. Any number of the CsgG pore monomers in the pore or pore complex, such as 6, 7, 8, 9 or 10, may be a variant of SEQ ID NO: 59. All six to ten monomers in the pore or pore complex are preferably variants of SEQ ID NO: 59. The variants in the pore complex may be the same or different. The variants are preferably identical in each pore monomer conjugate in the pore complex.

### Linkers

In some embodiments, a protein pore complex is stabilized by attachment (e.g., covalent attachment) of an auxiliary protein or fusion protein to the nanopore. The covalent linkage may for example be a disulphide bond, or click chemistry. By way of further example, cysteine residues may be connected by means of a linker such as BMOE. The auxiliary protein or fusion protein and/or the transmembrane protein nanopore may be modified to facilitate such covalent interactions. In some embodiments, the auxiliary protein or fusion protein is non-covalently attached to the nanopore. In some embodiments, the auxiliary protein or fusion protein is attached to the nanopore by one or more (e.g., 1, 2, 3, 4, 5, or more) linkers.

In some embodiments, an auxiliary protein or fusion protein is attached to a nanopore by hydrophobic interactions and/or by one or more disulfide bond. One or more, such as 2, 3, 4, 5, 6, 8, 9, for example all, of the monomers in a pore may be modified to enhance such interactions. This may be achieved in any suitable way. Further suitable interactions include salt bridges, electrostatic interactions, formation of hydrogen bonds, peptide bond formation, and Pi-Pi interactions.

At least one cysteine residue in the amino acid sequence of the transmembrane protein nanopore at the interface between the nanopore and auxiliary protein (or fusion protein) may be disulfide bonded to at least one cysteine residue in the amino acid sequence of the auxiliary protein at the interface between the nanopore and auxiliary protein. In some embodiments, at least one cysteine residue in the amino acid sequence of a first auxiliary protein is disulfide bonded to at least one cysteine residue in the amino acid sequence of a second auxiliary protein. In some embodiments, at least one cysteine residue in the amino acid sequence of a first portion of a fusion protein is disulphide bonded to at least one cysteine residue in the amino acid sequence of a second portion of the fusion protein. The cysteine residue in the nanopore and/or the cysteine residue in the auxiliary protein or fusion protein may be a cysteine residue that is not present in the wild type transmembrane protein pore monomer or in the wild-type auxiliary protein. Multiple disulfide bonds, such as from 2, 3, 4, 5, 6, 7, 8 or 9 to 16, 18, 24, 27, 32, 36, 40, 45, 48, 54, 56 or 63, may form between the nanopore and auxiliary protein (or fusion protein) in the pore complex. One or both of the nanopore and the auxiliary protein (or fusion protein) may comprise at least one monomer, or subunit, such as up to 8, 9 or 10 monomers or subunits, that comprises a cysteine residue at the interface between the nanopore and auxiliary protein (or fusion protein).

The nanopore and/or auxiliary protein (or fusion protein) may comprise one or more hydrophobic amino acid residue at the interface between the nanopore and auxiliary protein (or fusion protein), which is more hydrophobic than the residue present at the corresponding position in the wild type nanopore or auxiliary protein (or fusion protein). At least one monomer, or subunit, in the nanopore and/or at least one monomer, or subunit, in the auxiliary protein (or fusion protein) may comprise at least one residue at the interface between the nanopore and auxiliary protein (or fusion protein), which residue is more hydrophobic than the residue present at the corresponding position in the wild type pore or auxiliary protein (or fusion protein). For example, from 2 to 10, such as 3, 4, 5, 6, 7, 8 or 9, residues in the nanopore and/or the auxiliary protein (or fusion protein) may be more hydrophobic that the residues at the same positions in the corresponding wild type nanopore and/or the auxiliary protein (or fusion protein). Such hydrophobic residues strengthen the interaction between the nanopore and the auxiliary protein (or fusion protein) in the pore complex. Where the residue at the interface in the wild type nanopore or auxiliary protein (or fusion protein) is R, Q, N or E, the hydrophobic residue is typically I, L, V, M, F, W, A, or Y. Where the residue at the interface in the wild type nanopore or auxiliary protein (or fusion protein) is I, the hydrophobic residue is typically L, V, M, F, W, A, or Y. Where the residue at the interface in the wild type nanopore or auxiliary protein (or fusion protein) is L, the hydrophobic residue is typically I, V, M, F, W, A, or Y.

Molecular dynamics simulations can be performed to establish which residues in the auxiliary protein and nanopore come into close proximity. This information can be used to design auxiliary protein and/or transmembrane protein nanopore mutants that could increase the stability of the complex. For example, simulations can be performed using the GROMACS package version 4.6.5, with the GROMOS 53a6 force field and the SPC water model using cryo-EM structure of the proteins. The complex can be solvated and then energy minimized using the steepest descents algorithm. Throughout the simulation, restraints can be applied to the backbones of the proteins, however, the residue side chains can be free to move. The system can be simulated in the NPT ensemble for 20 ns, using the Berendsen thermostat and Berendsen barostat to 300 K. Contacts between the auxiliary protein and nanopore can be analysed using GROMACS analysis software and/or locally written code. Two residues can be defined as having made a contact if they come within 3 Angstroms of each other.

For example, in a pore complex, the interaction between a CsgF peptide and a CsgG pore may, for example, be stabilized by hydrophobic interactions, electrostatic interactions, or a covalent bond at a position corresponding to one or more of the following pairs of positions of SEQ ID NO: 60 and SEQ ID NO: 59, respectively: 1 and 153, 4 and 133, 5 and 136, 8 and 187, 8 and 203, 9 and 203, 11 and 142, 11 and 201, 12 and 149, 12 and 203, 26 and 191, 29 and 144, or 30 and 196. The residues in CsgF and/or CsgG at one or more of these positions may be modified in order to enhance the interaction between CsgG and CsgF in the pore.

The covalent link or binding is, for example, via cysteine linkage, wherein the sulfhydryl side group of cysteine covalently links with another amino acid residue or moiety and/or via an interaction between non-native (photo) reactive amino acids. (Photo-) reactive amino acids are referring to artificial analogs of natural amino acids that can be used for crosslinking of protein complexes, and may be incorporated into proteins and peptides in vivo or in vitro. Photo-reactive amino acid analogs in common use are photoreactive diazirine analogs to leucine and methionine, and para-benzoyl-phenyl-alanine, as well as azidohomoalanine, homopropargylglycyine, homoallelglycine, p-acetyl-Phe, p-azido-Phe, p-propargyloxy-Phe and p-benzoyl-Phe (Wang et al. 2012; Chin et al. 2002). Upon exposure to ultraviolet light, they are activated and covalently bind to interacting proteins that are within a few angstroms of the photo-reactive amino acid analog.

The pore complex can be made and disulphide bond formation can be induced by using oxidising agents (eg: Copper-orthophenanthroline). Other interactions (eg: hydrophobic interactions, charge-charge interactions/electrostatic interactions) can also be used in those positions instead of cysteine interactions. In another embodiment, unnatural amino acids can also be incorporated in those positions. In this embodiment, covalent bonds made be made by via click chemistry. For example, unnatural amino acids with azide or alkyne or with a dibenzocyclooctyne (DBCO) group and/or a bicyclo[6.1.0]nonyne (BCN) group may be introduced at one or more of these positions.

For example, the CsgG pore may comprise at least one, such as 2, 3, 4, 5, 6, 7, 8, 9 or 10, CsgG monomers that is/are modified to facilitate attachment to an auxiliary protein or fusion protein. For example, a cysteine residue may be introduced at one or more of the positions corresponding to positions 132, 133, 136, 138, 140, 142, 144, 145, 147, 149, 151, 153, 155, 183, 185, 187, 189, 191, 201, 203, 205, 207 and 209 of SEQ ID NO: 59, and/or at any position being predicted to make contact with the auxiliary protein or fusion protein, to facilitate covalent attachment to the auxiliary protein or fusion protein. As an alternative or addition to covalent attachment via cysteine residues, the pore may be stabilized by hydrophobic interactions or electrostatic interactions. To facilitate such interactions, a non-native reactive or photoreactive amino acid at a position corresponding to one or more of positions 132, 133, 136, 138, 140, 142, 144, 145, 147, 149, 151, 153, 155, 183, 185, 187, 189, 191, 201, 203, 205, 207 and 209 of SEQ ID NO: 59.

For example, a CsgF peptide may be modified to facilitate attachment to the CsgG pore. For example, a cysteine residue may be introduced at one or more of the positions corresponding to positions 1, 4, 5, 8, 9, 11, 12, 26 or 29 of SEQ ID NO: 60, and/or any position as being predicted to make contact with CsgG, to facilitate covalent attachment to CsgG. As an alternative or addition to covalent attachment via cysteine residues, the pore may be stabilized by hydrophobic interactions or electrostatic interactions. To facilitate such interactions, a non-native reactive or photoreactive amino acid at a position corresponding to one or more of positions 1, 2, 3, 4, 5, 8, 9, 11, 12, 26 or 29 of SEQ ID NO: 60.

Such stabilizing mutations can be combined with any other modifications to the auxiliary protein or fusion protein, for example the modifications to improve the interaction of the pore complex with a polynucleotide, or to improve certain properties of the complex (e.g., discrimination of polymer units, such as nucleotides of a polynucleotide).

In some embodiments, a nanopore may be isolated, substantially isolated, purified or substantially purified. A pore is isolated or purified if it is completely free of any other components, such as lipids or other pores. A pore is substantially isolated if it is mixed with carriers or diluents which will not interfere with its intended use. For instance, a pore is substantially isolated or substantially purified if it is present in a form that comprises less than 10%, less than 5%, less than 2% or less than 1% of other components, such as block copolymers, lipids or other pores. Alternatively, the pore may be present in a membrane. Suitable membranes are discussed below.

The pore complex of may be present in a membrane as an individual or single pore. Alternatively, the pore complex may be present in a homologous or heterologous population of two or more pores.

The auxiliary protein or fusion may be attached directly to the transmembrane protein nanopore, or the two proteins (e.g., a first auxiliary protein and a second auxiliary protein; a first portion of a fusion protein and a second portion of a fusion protein, etc.) may be attached using a linker, such as a chemical crosslinker or a peptide linker.

Suitable chemical crosslinkers are well-known in the art. Examples of crosslinkers include but are not limited to 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. In some embodiments, the crosslinker is succinimidyl 3-(2-pyridyldithio)propionate (SPDP). Typically, the molecule is covalently attached to the bifunctional crosslinker before the molecule/crosslinker complex is covalently attached to the mutant monomer but it is also possible to covalently attach the bifunctional crosslinker to the monomer before the bifunctional crosslinker/monomer complex is attached to the molecule. In some embodiments, the linker is resistant to dithiothreitol (DTT). Additional suitable linkers include, but are not limited to, iodoacetamide-based and Maleimide-based linkers.

Suitable amino acid linkers, such as peptide linkers, are known in the art. The length, flexibility and hydrophilicity of the amino acid or peptide linker are typically designed such that the auxiliary protein or fusion protein forms a constriction in the pore complex. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P)₁₂ wherein P is proline.

Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Linkers can vary in length from one carbon (phosgene-type linkers) to many Angstroms. Examples of linker molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides. These linkers may be inert or reactive, in particular they may be chemically cleavable at a defined position, or may be themselves modified with a fluorophore or ligand. The linker is preferably resistant to dithiothreitol (DTT) following the covalent attachment of the auxiliary protein or fusion protein to the CsgG pore monomer.

In some embodiments, the crosslinker is selected from: 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate, di-maleimide PEG 1k, di-maleimide PEG 3.4k, di-maleimide PEG 5k, di-maleimide PEG 10k, bis(maleimido)ethane (BMOE), bis-maleimidohexane (BMH), 1,4-bis-maleimidobutane (BMB), 1,4 bis-maleimidyl-2,3-dihydroxybutane (BMDB), BM[PEO]2 (1,8-bis-maleimidodiethyleneglycol), BM[PEO]3 (1,11-bis-maleimidotriethylene glycol), tris[2-maleimidoethyl]amine (TMEA), DTME dithiobismaleimidoethane, bis-maleimide PEG3, bis-maleimide PEG11, DBCO-maleimide, DBCO-PEG4-maleimide, DBCO-PEG4-NH2, DBCO-PEG4-NHS, DBCO-NHS, DBCO-PEG-DBCO 2.8 kDa, DBCO-PEG-DBCO 4.0 kDa, DBCO-15 atoms-DBCO, DBCO-26 atoms-DBCO, DBCO-35 atoms-DBCO, DBCO-PEG4-S-S-PEG3-biotin, DBCO-S-S-PEG3-biotin, DBCO-S-S-PEG11-biotin, (succinimidyl 3-(2-pyridyldithio) propionate (SPDP) and maleimide-PEG(2 kDa)-maleimide (ALPHA,OMEGA-BIS-MALEIMIDO POLY(ETHYLENE GLYCOL)). In some embodiments, the crosslinker is maleimide-propyl-SRDFWRS-(1,2-diaminoethane)-propyl-maleimide.

The linked CsgG pore monomer and auxiliary protein or fusion protein may be coupled via the formation of covalent bonds between the groups. Any of the specific linkers disclosed in WO 2010/086602 (incorporated herein by reference in its entirety) may be used.

The linkers may be labelled. Suitable labels include, but are not limited to, fluorescent molecules (such as Cy3 or AlexaFluor®555), radioisotopes, e.g., ¹²⁵I, ³⁵S, ³²P, enzymes, antibodies, antigens, polynucleotides, and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting the pore monomer conjugates is via cysteine linkage. This can be mediated by a bi-functional chemical crosslinker or by an amino acid linker with a terminal presented cysteine residue.

Another preferred method of attachment via 4-azidophenylalanine (Faz) linkage. This can be mediated by a bi-functional chemical linker or by a polypeptide linker with a terminal presented Faz residue.

In some embodiments, the linker is a bond formed by a Sulfur (VI) fluoride exchange (SuFEx) reaction. In some embodiments, an auxiliary protein (e.g., a CsgF or portion of CsgF) can be functionalized with a sulphonyl fluoride group which when in the right proximity can react with a nucleophilic amino acid (e.g., a nucleophilic amino acid of a CsgG pore monomer, a nucleophilic acid of another auxiliary protein, etc.) to form a sulphonyl bond (SuFEX).

The auxiliary protein or fusion protein may be genetically fused to the transmembrane protein nanopore. The pore monomer and auxiliary protein (or fusion protein) are genetically fused if the whole construct is expressed from a single polynucleotide coding sequence. The monomer, or subunit, auxiliary protein (or fusion protein) may be directly fused to a monomer, or subunit, of the transmembrane protein nanopore. Alternatively, the monomer, or subunit, auxiliary protein (or fusion protein) may be fused to a monomer, or subunit, of the transmembrane protein nanopore via one or more linkers.

The distance between the CsgG pore monomer and the auxiliary protein or fusion protein in the CsgG pore monomer conjugate and/or the length of the linker is preferably less than about 2.00 nm, such as less than about 1.90 nm, less than about 1.80 nm, less than about 1.70 nm, less than about 1.60 nm, less than about 1.50 nm, less than about 1.40 nm, less than about 1.30 nm, less than about 1.20 nm, less than about 1.10 nm, less than about 1.00 nm, less than about 0.90 nm, less than about 0.80 nm, less than about 0.70 nm, less than about 0.60 nm, less than about 0.50 nm, or less than about 0.40 nm. The distance between the CsgG pore monomer and the auxiliary protein or fusion protein in the pore monomer conjugate and/or the length of the linker is preferably less than about 1.20 nm. This distance/length can be achieved using maleimidohexanonic acid as discussed in more detail below. The distance between the CsgG pore monomer and the auxiliary protein or fusion protein in the pore monomer conjugate and/or the length of the linker is preferably less than about 0.8 nm. This distance/length can be achieved using maleimidopropionic acid as discussed below.

The distance between the CsgG pore monomer and the auxiliary protein or fusion protein in the pore monomer conjugate and/or the length of the linker is preferably from about 0.40 nm to about 2.0 nm, such as about 0.45 nm to about 1.90 nm, from about 0.50 nm to about 1.80 nm, from about 0.55 nm to about 1.7 nm, from about 0.60 nm to about 1.6 nm, from about 0.65 nm to about 1.5 nm, from about 0.7 nm to about 1.4 nm, from about 0.75 nm to about 1.3 nm, from about 0.80 nm to about 1.2 nm, from about 0.85 nm to about 1.1 nm and from about 0.90 nm to about 1.00 nm. The distance between the CsgG pore monomer and the auxiliary protein or fusion protein in the pore monomer conjugate and/or the length of the linker is preferably from about 0.50 nm to about 1.50 nm. The distance between the CsgG pore monomer and the auxiliary protein or fusion protein in the pore monomer conjugate and/or the length of the linker is preferably from about 0.60 nm to about 1.2 nm. This distance/length can be achieved using any of specific maleimide-containing linkers discussed below.

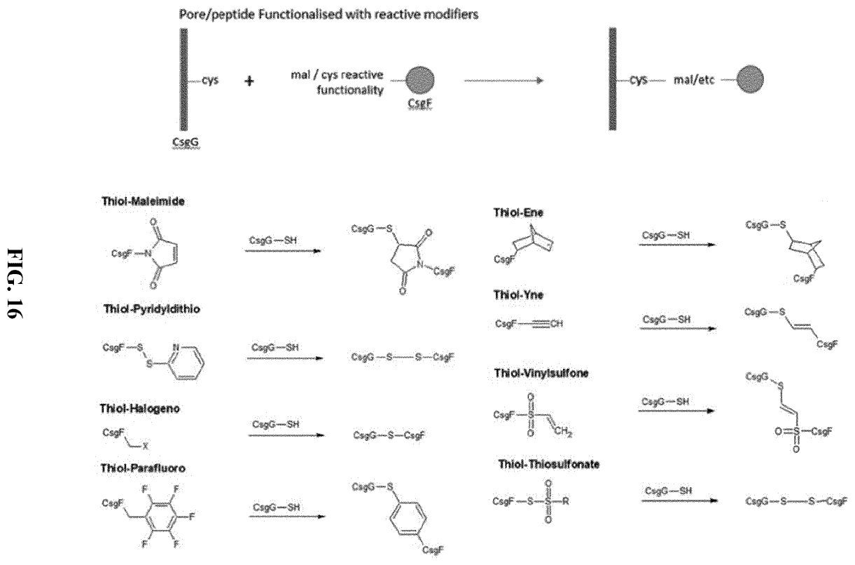

The maleimide-containing linker may be any of the linkers discussed below with reference to the constructs described herein. The maleimide-containing linker preferably comprises or consists of a maleimide group and a linear carbon chain of 2, 3, 4, 5, 6 or more carbon atoms. The linear carbon chain is typically attached to the nitrogen atom in the maleimide group. The linear carbon chain also preferably comprises a terminal carboxyl group. This carboxyl group is capable of forming an amide bond with an amino acid in the auxiliary protein or fusion protein. The linker is preferably maleimidoacetic acid, maleimidopropionic acid, maleimidobutyric acid, maleimidopentanoic acid or maleimidohexanonic acid. The linker is most preferably maleimidopropionic acid. This linker is shown in FIG. 15.

The disclosure also provides a pore monomer conjugate comprising a CsgG pore monomer covalently attached to an auxiliary protein or fusion protein, wherein the auxiliary protein or fusion protein is covalently attached to a cysteine residue in the CsgG pore monomer by a linker comprising a thiol-reactive group. The thiol reactive group may be a maleimide group, pyridyldithio group, halogeno group, parafluoro group, ene group, yne group, vinylsulfone group or thiosulfone group. These groups are shown in FIG. 16. The linker comprising a thiol-reactive group may be any of the linkers discussed below with reference to the constructs of the disclosure. The linker preferably comprises or consists of the thiol reactive group and a linear carbon chain of 2, 3, 4, 5, 6 or more carbon atoms. The linear carbon chain also preferably comprises a terminal carboxyl group. This carboxyl group is capable for forming an amide bond with an amino acid in the auxiliary protein or fusion protein. The linker may be any of the specific maleimide-containing linkers discussed above with the maleimide replaced by a different thiol-reactive group. The linker containing the thiol-reactive group may be any of the lengths discussed above.

Appropriate linking groups may be designed using conventional modelling techniques. The linker is typically sufficiently flexible to allow the monomers, or subunits, to assemble into their respective protein oligomers, and to align along their common symmetry axis in order to produce a continuous channel within the pore complex.

### Identification and Selection of Auxiliary Proteins

Aspects of the disclosure relate to computer-based methods of designing and/or selecting auxiliary proteins and/or fusion proteins for inclusion into protein pore complexes (e.g., protein pore complexes comprising a CsgG nanopore). In some embodiments, the methods comprise providing an amino acid sequence (e.g., a CsgF amino acid sequence) as input to software comprising code that implements a protein backbone sequence selection technique and processes the amino acid sequence to produce a backbone amino acid sequence as output. In some embodiments, the protein backbone selection technique may be MASTER (e.g., as described by Zhou and Grigoryan, Protein Sci. 2015 April; 24 (4): 508-524, the entire contents of which are incorporated herein by reference). In some embodiments, the protein backbone selection technique comprises selecting, from known protein backbone structure (e.g., as described in the Protein Data Bank, PDB), a protein backbone structure having one or more target characteristics (e.g., ability to form one or more helical regions, ability to pack with one or more helical regions of a protein pore, etc.). In some embodiments, the backbone structure is provided as input to software comprising code that implements a protein sequence design and structural prediction technique and processes the backbone structure to produce one or more de novo designed peptide sequences. In some embodiments, the protein sequence design and structural prediction technique may be Rosetta (e.g., as described by Leaver-Fay et al. Chapter nineteen—Rosetta3: An Object-Oriented Software Suite for the Simulation and Design of Macromolecules, Methods in Enzymology, Academic Press, Volume 487, 2011, pages 545-574, doi.org/10.1016/B978-0-12-381270-4.00019-6, the entire contents of which are incorporated herein by reference). In some embodiments, the de novo designed peptide sequences comprise one or more target characteristics that are the same as the one or more desirable characteristics of the backbone amino acid sequence.

### Methods of Producing Nanopore Complexes

The pore complex comprising an auxiliary protein or fusion protein and a transmembrane protein nanopore can, in one embodiment, be made via co-expression. In some embodiments, the method comprises the steps of expressing both pore monomers and the auxiliary protein or fusion protein, or auxiliary proteins or monomers, in a suitable host cell, and allowing in vivo complex pore formation. In this embodiment, at least one gene encoding a pore monomer in one vector and a gene encoding the auxiliary protein or fusion protein, or at least one auxiliary protein subunit or monomer in a second vector may be transformed together to express the proteins and make the complex within transformed cells. This is preferably carried out ex vivo or in vitro. Alternatively, the two genes encoding the pore monomer and auxiliary protein (or fusion protein), or subunit thereof, can be placed in one vector under the control of a single promotor or under the control of two separate promoters, which may be the same or different.

Another method for producing the pore complex formed by the auxiliary protein or fusion protein and a transmembrane protein nanopore is in vitro reconstitution of proteins to obtain a functional pore. In some embodiments, the method comprises the steps of contacting the monomers of the transmembrane protein nanopore, with the auxiliary protein (or fusion protein), or auxiliary protein subunits or monomers, in a suitable system to allow complex formation. Said system may be an “in vitro system”, which refers to a system comprising at least the necessary components and environment to execute said method, and makes use of biological molecules, organisms, a cell (or part of a cell) outside of their normal naturally-occurring environment, permitting a more detailed, more convenient, or more efficient analysis than can be done with whole organisms. An in vitro system may also comprise a suitable buffer composition provided in a test tube, wherein said protein components to form the complex have been added. A person skilled in the art is aware of the options to provide said system.

In this embodiment, the nanopore may be produced by expressing the monomer(s) separately from the auxiliary protein or fusion protein. Pore monomers or a nanopore may be purified from the cells transformed with a vector encoding at least one pore monomer, or with more than one vector each expressing a pore monomer. The auxiliary protein or fusion protein may be purified from the cells transformed with a vector encoding at least one auxiliary protein or fusion protein. The purified pore monomer(s)/nanopore may then be incubated together with the auxiliary protein or fusion protein to make the pore complex.

In another embodiment, the nanopore monomer(s) and/or the auxiliary protein or fusion protein are produced separately by in vitro translation and transcription (IVTT). The nanopore monomer(s) may then be incubated together with the auxiliary protein or fusion protein to make the pore complex.

The above embodiments may be combined, such that for example, (i) the nanopore is produced in vivo and the auxiliary protein or fusion protein in vivo; (ii) the nanopore is produced in vitro and the auxiliary protein or fusion protein in vivo; (iii) the nanopore is produced in vivo and the auxiliary protein or fusion protein in vitro; or (iv) the nanopore is produced in vitro and the auxiliary protein or fusion protein in vitro.

One or both of the nanopore monomer and the auxiliary protein or fusion protein may be tagged to facilitate purification. Purification can also be performed when the nanopore monomer and/or auxiliary protein or fusion protein are untagged. Methods known in the art (e.g. ion exchange, gel filtration, hydrophobic interaction column chromatography etc.) can be used alone or in different combinations to purify the components of the pore complex.

Any known tags can be used in any of the two proteins. In one embodiment, two tag purification can be used to purify the pore complex from its component parts. For example, a Strep tag can be used in the nanopore and His tag can be used in the auxiliary protein (or fusion protein) or vice versa. A similar end result can be obtained when the two proteins are purified individually and mixed together followed by another round of Strep and His purification.

The pore complex can be made prior to insertion into a membrane or after insertion of the nanopore into a membrane. However, the nanopore may be inserted into a membrane and the auxiliary protein (or fusion protein) may be added afterwards so that the pore complex can form in situ. For example, in one embodiment, a system where the trans side or cis side of the membrane is accessible (for example in a chip or chamber for electrophysiology measurements), the nanopore may be inserted into the membrane, and then an auxiliary protein (or fusion protein) may be added from the trans side or cis side of the membrane, so that the complex can be formed in-situ.

In one embodiment, the auxiliary protein may comprise a protease cleavage site (e.g. TEV, HRV 3 or any other protease cleavage site), and be cleaved before or after associating with the nanopore. For example, a full length auxiliary protein (or fusion protein) may be used to form the pore. Cleavage of amino acid residues that do not form part of the channel construction and are not required for interaction with the transmembrane pore may be cleaved from the auxiliary protein or fusion protein. In this embodiment, once the pore complex is formed, the protease is used to cleave the auxiliary protein or fusion protein. Alternatively, the protease may be used to produce the auxiliary protein or fusion protein prior to pore complex assembly.

Some protease sites will leave an additional tag (or a portion thereof, for example one or more amino acids of the tag) behind after cleavage. For example, the TEV protease cleavage sequence is ENLYFQS. TEV protease cleaves the protein between Q and S leaving ENLYFQ intact at the C-terminus of the CsgF peptide. By way of another example, the HRV C3 cleavage site is LEVLFQGP and the enzyme cleaves between Q and G leaving LEVLFQ intact at the C-terminus of the CsgF peptide.

The protein may be chemically modified with a molecular adaptor that facilitates the interaction between a pore comprising the monomer and a target nucleotide or target polynucleotide sequence. Suitable adaptors, including a cyclic molecule, a cyclodextrin, a species that is capable of hybridization, a DNA binder or interchelator, a peptide or peptide analogue, a synthetic polymer, an aromatic planar molecule, a small positively charged molecule or a small molecule capable of hydrogen-bonding, are described in WO 2019/002893 (incorporated by reference herein in its entirety). The molecular adaptor may be attached using any of the methods and linkers discussed above.

The protein may be attached to a polynucleotide binding protein. This forms a modular sequencing system. Polynucleotide binding proteins are discussed below. The protein can be covalently attached to the monomer using any method known in the art. The monomer and protein may be chemically fused or genetically fused. Genetic fusion of a monomer to a polynucleotide binding protein is discussed in WO 2010/004265 (incorporated herein by reference in its entirety). The polynucleotide binding protein may be attached via cysteine linkage using any method described above.

The polynucleotide binding protein may be attached directly to the protein via one or more linkers. The molecule may be attached to the CsgG pore monomer using the hybridization linkers described in as WO 2010/086602 (incorporated herein by reference in its entirety). Alternatively, peptide linkers may be used. Suitable peptide linkers are discussed above.

Any of the proteins can be produced using standard methods known in the art. Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system and the Gilson HPLC system.

### System

In another aspect, the disclosure relates to a system for characterising a target polynucleotide, the system comprising a membrane and a pore complex; wherein the pore complex comprises: (i) a nanopore located in the membrane, and (ii) an auxiliary protein or fusion protein attached to the nanopore; wherein the nanopore and the auxiliary protein or fusion protein together form a continuous channel across the membrane, the channel comprising a first constriction region and a second constriction region.

The pore complex, nanopore and auxiliary protein or fusion protein may be any as described herein above.

In one embodiment, the system further comprises a first chamber and a second chamber, wherein the first and second chambers are separated by the membrane. When used to characterise a target polynucleotide, the system may further comprise a target polynucleotide, wherein the target polynucleotide is transiently located within the continuous channel and wherein one end of the target polynucleotide is located in the first chamber and one end of the target polynucleotide is located in the second chamber.

In one embodiment, the system further comprises an electrically-conductive solution in contact with the nanopore, electrodes providing a voltage potential across the membrane, and a measurement system for measuring the current through the nanopore. In one embodiment, the voltage applied across the membrane and pore complex is from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different nucleotides by a pore by using an increased applied potential. Any suitable electrically-conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g. in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

A buffer may be present in the electrically-conductive solution. Typically, the buffer is phosphate buffer. Other suitable buffers are HEPES and Tris-HCl buffer. The pH of the electrically-conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 6.9.

The system may comprise an array of pore complexes present in membranes. In a preferred embodiment, each membrane in the array comprises one pore complex. Due to the manner in which the array is formed, for example, the array may comprise one or more membrane that does not comprise a pore complex, and/or one or more membrane that comprises two or more pore complexes. The array may comprise from about 2 to about 12,000, such as from about 10 to about 800, from about 20 to about 600, from about 30 to about 500, from about 250 to about 2000, from about 500 to about 4000, from about 1000 to about 5000, from about 2500 to about 10,000, or from about 5000 to about 12,000 membranes. In some embodiments, an array comprises more than 12,000 membranes.

The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane containing the pore is formed. Alternatively, the barrier forms the membrane in which the pore is present.

In one embodiment, the apparatus comprises a sensor device that is capable of supporting the plurality of pores and membranes and being operable to perform analyte characterisation using the pores and membranes; and at least one port for delivery of the material for performing the characterisation.

In one embodiment, the apparatus comprises a sensor device that is capable of supporting the plurality of pores and membranes being operable to perform analyte characterisation using the pores and membranes; and at least one reservoir for holding material for performing the characterisation.

In one embodiment, the apparatus comprises a sensor device that is capable of supporting the membrane and plurality of pores and membranes and being operable to perform analyte characterising using the pores and membranes; at least one reservoir for holding material for performing the characterising; a fluidics system configured to controllably supply material from the at least one reservoir to the sensor device; and one or more containers for receiving respective samples, the fluidics system being configured to supply the samples selectively from one or more containers to the sensor device.

The apparatus may also comprise an electrical circuit capable of applying a potential and measuring an electrical signal across the membrane and pore complex. The apparatus may be any of those described in WO 2008/102120, WO 2009/077734, WO 2010/122293, WO 2011/067559 or WO 00/28312.

### Membranes

Any suitable membrane may be used in the system. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e. lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. The membrane is preferably a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example, a hydrophobic polymer may be made from siloxane or other non-hydrocarbon based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer based membranes for a wide range of applications.

The membrane is most preferably one of the membranes disclosed in International Application No. WO2014/064443 or WO2014/064444.

The amphiphilic molecules may be chemically-modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording.

Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

Methods for forming lipid bilayers are known in the art. Lipid bilayers are commonly formed by the method of Montal and Mueller (Proc. Natl. Acad. Sci. USA., 1972; 69:3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed. Planar lipid bilayers may be formed across an aperture in a membrane or across an opening into a recess.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847). In a preferred embodiment, the lipid bilayer is formed as described in International Application No. WO 2009/077734. Advantageously in this method, the lipid bilayer is formed from dried lipids. In a most preferred embodiment, the lipid bilayer is formed across an opening as described in WO2009/077734.

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester. The lipids may be mycolic acid.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy (Polyethylene glycol)-2000]; functionalised PEG Lipids, such as 1,2-Distearoyl-sn-Glycero-3 Phosphoethanolamine-N-[Biotinyl(Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine. The lipids may be chemically-modified or functionalised to facilitate coupling of the polynucleotide.

The amphiphilic layer, for example the lipid composition, typically comprises one or more additives that will affect the properties of the layer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides.

In another preferred embodiment, the membrane comprises a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The method is typically carried out using (i) an artificial amphiphilic layer comprising a pore, (ii) an isolated, naturally-occurring lipid bilayer comprising a pore, or (iii) a cell having a pore inserted therein. The method is typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the disclosure is typically carried out in vitro.

### Methods of Characterising Analytes

In a further aspect, a method of determining the presence, absence or one or more characteristics of a target analyte is disclosed. The method involves contacting the target analyte with a membrane comprising a pore complex, such that the target analyte moves with respect to, such as into or through, the continuous channel comprising at least two constructions provided by a nanopore and an auxiliary protein or peptide in the pore complex, respectively, and taking one or more measurements as the analyte moves with respect to the channel and thereby determining the presence, absence or one or more characteristics of the analyte. The analyte may pass through the nanopore constriction, followed by the auxiliary protein constriction. In an alternative embodiment the analyte may pass through the auxiliary protein constriction, followed by the nanopore constriction, depending on the orientation of the pore complex in the membrane.

In one embodiment, the method is for determining the presence, absence or one or more characteristics of a target analyte. The method may be for determining the presence, absence or one or more characteristics of at least one analyte. The method may concern determining the presence, absence or one or more characteristics of two or more analytes. The method may comprise determining the presence, absence or one or more characteristics of any number of analytes, such as 2, 5, 10, 15, 20, 30, 40, 50, 100 or more analytes. Any number of characteristics of the one or more analytes may be determined, such as 1, 2, 3, 4, 5, 10 or more characteristics.

The binding of a molecule in the channel of the pore complex, or in the vicinity of either opening of the channel will have an effect on the open-channel ion flow through the pore, which is the essence of “molecular sensing” of pore channels. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for nanopore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalized drug development, medicine, diagnostics, life science research, environmental monitoring and in the security and/or the defense industry.

The target analyte may be a metal ion, an inorganic salt, a polymer, an amino acid, a peptide, a polypeptide, a protein, a nucleotide, an oligonucleotide, a polynucleotide, a monosaccharide, a polysaccharide, a dye, a bleach, a pharmaceutical, a diagnostic agent, a recreational drug, an explosive, a toxic compound, or an environmental pollutant. The method may concern determining the presence, absence or one or more characteristics of two or more analytes of the same type, such as two or more proteins, two or more nucleotides or two or more pharmaceuticals. Alternatively, the method may concern determining the presence, absence or one or more characteristics of two or more analytes of different types, such as one or more proteins, one or more nucleotides and one or more pharmaceuticals.

The target analyte can be secreted from cells. Alternatively, the target analyte can be an analyte that is present inside cells such that the analyte must be extracted from the cells before the method can be carried out.

In one embodiment, the analyte is an amino acid, a peptide, a polypeptides or protein. The amino acid, peptide, polypeptide or protein can be naturally-occurring or non-naturally-occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

In a preferred embodiment, the analyte is a polynucleotide, such as a nucleic acid. A polynucleotide is defined as a macromolecule comprising two or more nucleotides. The naturally-occurring nucleic acid bases in DNA and RNA may be distinguished by their physical size. As a nucleic acid molecule, or individual base, passes through the channel of a nanopore, the size differential between the bases causes a directly correlated reduction in the ion flow through the channel. The variation in ion flow may be recorded. Suitable electrical measurement techniques for recording ion flow variations are described in, for example, WO 2000/28312 and D. Stoddart et al., Proc. Natl. Acad. Sci., 2010, 106, pp 7702-7 (single channel recording equipment); and, for example, in WO 2009/077734 (multi-channel recording techniques). Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, it has typically required for the reduction in ion flow through the channel to be directly correlated to the size of the individual nucleotide passing through the constriction (or “reading head”). It will be appreciated that sequencing may be performed upon an intact nucleic acid polymer that is ‘threaded’ through the pore via the action of an associated polymerase or helicase, for example. Alternatively, sequences may be determined by passage of nucleotide triphosphate bases that have been sequentially removed from a target nucleic acid in proximity to the pore (see for example WO 2014/187924).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e. lack a nucleobase). A nucleotide may also lack a nucleobase and a sugar (i.e. is a C3 spacer). The sequence of the nucleotides (iii) is determined by the consecutive identity of following nucleotides attached to each other throughout the polynucleotide strain, in the 5′ to 3′ direction of the strand.

The pore complexes comprising at least two constrictions are particularly useful in analyzing homopolymers. For example, the pores may be used to determine the sequence of a polynucleotide comprising two or more, such as at least 3, 4, 5, 6, 7, 8, 9 or 10, consecutive nucleotides that are identical. For example, the pores may be used to sequence a polynucleotide comprising a polyA, polyT, polyG and/or polyC region.

In some embodiments, a CsgG pore constriction is made of the residues at the 51, 55 and 56 positions of SEQ ID NO: 59. When DNA is passing through the constriction, interactions of approximately 5 bases of DNA with the constriction of the pore at any given time dominate the current signal. Although certain CsgG pores (e.g., CsgG pores lacking one or more auxiliary proteins or fusion proteins as described herein) are very good in reading mixed sequence regions of DNA (when A, T, G and C are mixed), the signal becomes flat and lacks some information when there is a homopolymeric region within the DNA (eg: polyT, polyG, polyA, polyC). Because 5 bases dominate the signal of the CsgG and its constriction mutants, it is difficult to discriminate homopolymers longer than 5 without using additional dwell time information. However, if DNA is passing through a second constriction, more DNA bases will interact with the combined constrictions, increasing the length of the homopolymers that can be discriminated.

### Kits

In a further aspect, the present disclosure also provides a kit for characterising a target polynucleotide. The kit comprises the disclosed pore complex, and the components of a membrane. The membrane is preferably formed from the components. The pore complex is preferably present in the membrane, together forming a transmembrane pore complex channel. The kit may comprise components of any type of membranes, such as an amphiphilic layer or a triblock copolymer membrane. The kit may further comprise a polynucleotide binding protein, such as a nucleic acid handling enzyme, for example a polymerase or a helicase. The kit may further comprise one or more anchors, such as cholesterol, for coupling the polynucleotide to the membrane. The kit may further comprise one or more polynucleotide adaptors that can be attached to a target polynucleotide to facilitate characterisation of the polynucleotide. In one embodiment, the anchor, such as cholesterol, is attached to the polynucleotide adaptor. The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the disclosure or details regarding for which organism the method may be used. Finally, the kit may also comprise additional components useful in polynucleotide characterization.

It is to be understood that although particular embodiments, specific configurations as well as materials and/or molecules, have been discussed herein for engineered cells and methods according to the present disclosure, various changes or modifications in form and detail may be made without departing from the scope and spirit of this disclosure. The following examples are provided to better illustrate particular embodiments, and they should not be considered limiting the application. The application is limited only by the claims.

### EXAMPLES

### Example 1

To create a helical constriction de novo design was used to select a small protein domain that is well-folded and projects to the desired degree into the lumen of a nanopore. A number of programs can be used for this purpose. This example describes a workflow that uses the program MASTER to facilitate backbone design, and Rosetta with variable backbone geometry for sequence selection.

To create a new domain projecting into the pore lumen programs such as RF-diffusion, CHROMA or the program MASTER can be used. Here we used MASTER. The Protein Data Bank (PDB) was searched for structures that match the following criteria 1) stabilization of the target region of CsgF (residues 16-30); 2) projection into the nanopore to create a new constriction between 10 Å and 30 Å in diameter (Ca to Ca distances of amino acid residues extending furthest into the pore lumen) when all units are generated using a 9-fold symmetry operator; 3) the new domain should not clash with any atoms in CsgG or symmetry mates from CsgF.

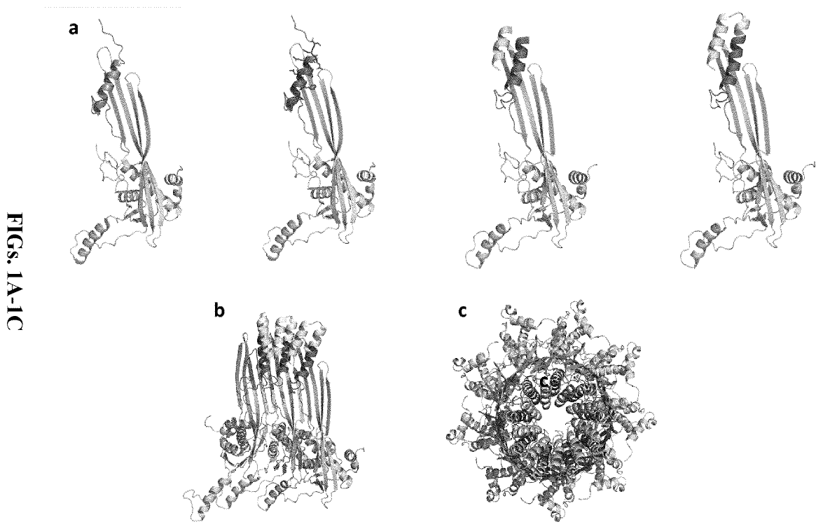

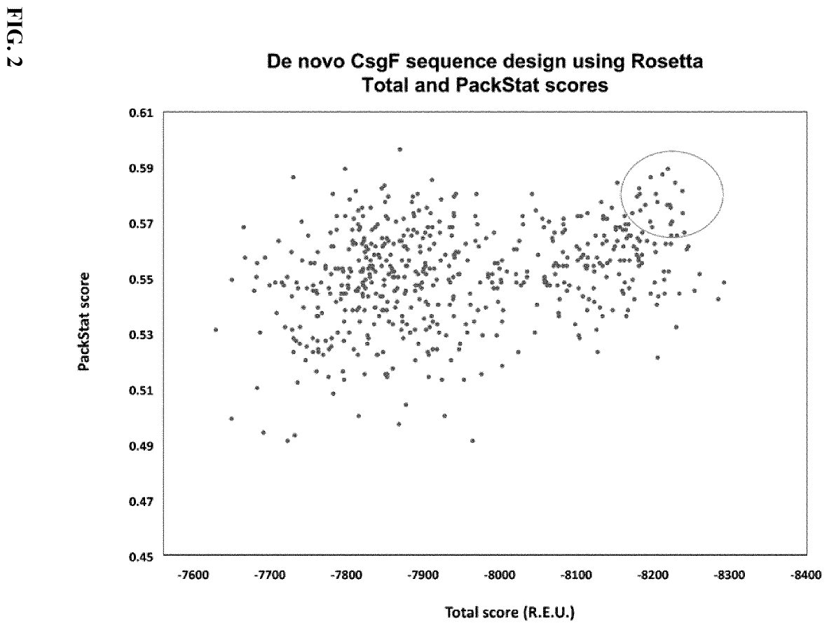

First, helices that would dock against the target region in CsgF and its symmetry neighbors, in a geometry that is frequently observed in natural proteins in the PDB, and hence is “designable” were identified. Top candidates were selected based on the number of the closely related helix-helix pairs found in the database after clustering of the output based on RMSD of the target region plus the discovered helix (FIG. 1). In this way, a geometry for the helix that packed well against the target and the four amino acids N-terminal to it was selected. Additionally, helices that engaged in favorable helix-helix interactions with symmetry related partners were searched in the database. A linker (e.g., a loop structure) connecting the helices was selected using a database of helical backbones (FIG. 1). Sequences for the resulting backbone were then designed using Rosetta. Representative sequences were produced (e.g., SEQ ID NOs: 1-58).

Sequences for experimental validation were chosen based on the lowest energy score and highest PackStat score as shown in FIG. 2. To further prioritize sequences, aggregation propensity may also be tested, using one of a number of aggregation and amyloid prediction programs.

### Example 2

### Materials and Methods

E. coli CsgG Pore Production

Recombinant expression vectors encoding the CsgG variant nanopores with a C-terminal Strep affinity tag and ampicillin resistance gene were transformed into chemically competent E. coli cells. The cells were plated onto an LB agar plate containing appropriate antibiotics for selection and incubated overnight at 37 °C. LB Media with appropriate antibiotics was inoculated with a single colony from the agar plate and grown overnight at 37 °C with shaking. The culture was diluted into autoinduction media plus necessary antibiotics and incubated at 18 °C for 68 hours with shaking. The cells were harvested through centrifugation before being lysed and extracted into a buffer containing 1× Bugbuster extraction reagent (Merck 70921) and 0.1% DDM. The lysate was spun down and the pore was purified from the soluble extract using affinity chromatography, heat treatment and then size exclusion chromatography, selecting for oligomeric nanopores as judged by SDS-PAGE.

### CsgG/CsgF or Fusion Protein Complex Formation Protocol

CsgG-CsgF complexes were prepared from nanopores purified as above and chemically synthesized de novo fusion proteins with or without a maleimide modification. For the fusion proteins comprising cysteines, cyclisation of the fusion protein was achieved by cross-linking the thiols at appropriate cysteines. Nanopores were buffer exchanged into a pH 7.0 buffer free of reducing agents and incubated with 8× molar excess of peptide to CsgG monomer for 1 hour at 25 °C. The sample was then heated at 60 °C for 15 minutes followed by centrifugation to remove any precipitate and DTT was added to prevent any further reaction.

### SDS-PAGE Analysis

1 μg of complex and CsgG-only pore control was added to individual 0.5 mL ProteinLoBind Eppendorf tubes (Fisher, 10316752) and made to 10 μL volume with Reaction Buffer. This was made to a final volume of 20 μL by the addition of 10 μL of 2× Laemmli buffer. Each sample was loaded in its entirety on a 4-20% TGX gel (BioRad, 5671093) running with 1× TGS buffer (Sigma, T7777). This was run for 21 minutes at 300V. To image the gel, Spyro Ruby (Merk, S4942) stain was used as per the manufacturer's instructions. This was then imaged on a GE Typhoon gel imager using a 450 nm laser.

For some analyses, 1 μg of complex and CsgG-only pore control was added to individual PCR tubes and made to 10 μL volume with Reaction Buffer. A freshly prepared 1M DTT stock was prepared, and this was spiked into the individual PCR tubes at a final concentration of 10 mM. This was made to a final volume of 20 μL by the addition of 10 μL of 2× Laemmli buffer. Each sample was heated on a PCR thermocycler for 2 minutes at 95 °C. This was allowed to cool for 5 minutes, before the material from each sample was loaded in its entirety on a 4-20% TGX gel (BioRad, 5671093) running with 1×TGS buffer (Sigma, T7777). This was run for 21 minutes at 300V. To image the gel, Spyro Ruby (Merk, S4942) stain was used as per the manufacturer's instructions. This was then imaged on a GE Typhoon gel imager using a 450 nm laser.

### Electrical Measurements

Electrical measurements were acquired from CsgG-only, CsgG/CsgF, or CsgG/Fusion Protein complexes that were inserted into MinION flow cells. After a single pore inserted into the block co-polymer membrane, 1 mL of a buffer comprising 25 mM Potassium Phosphate, 150 mM Potassium Ferrocyanide (II), 150 mM Potassium Ferricyanide (III), pH 8.0 was flowed through the system to remove any excess nanopores.

The analyte being used to assess the DNA squiggle was a 3.6-kilobase DNA section from the 3′ end of the lambda genome, as described in FIG. 23. Preparation of the analyte, ligating the analyte to the Y-adapter, SPRI-bead clean-up of the ligated analyte and addition to a minION flow cell was carried out using the Oxford Nanopore Technologies Q-SQK-LSK110 protocol.

Electrical measurements were acquired using minION Mk1b from Oxford Nanopore Technologies. A standard sequencing script at −180 mV was run for 6 hours, with static flicks every 5 minute to remove extended nanopore blocks. Raw data was collected in a bulk FAST5 file using MinKNOW software (Oxford Nanopore Technologies).

### Discrimination Profiling

FAST5s containing DNA squiggles (e.g., electrical measurements) for the 3.6-kilobase DNA section from the 3′ end of the lambda genome (3.6 Kb lambda) were acquired. DNA squiggles were trimmed using custom python scripts to remove any electrical signal measurements that were captured before DNA sequencing was initiated.

Trimmed 3.6 Kb lambda squiggles and the corresponding genome reference for this region were used to train the parameters of a neural network. The neural network, containing 4 layers, modelled sequences of a user-specified window-length and the associated current level of those sequences. The window length specified for these models allowed for a region of +/−12 nucleotides to contribute to the current level at any one position.

The trained neural network was used to predict the current levels that would correspond to the 3.6 Kb lambda DNA reference sequence. It was also used to predict the current levels from all possible single-base edits of that sequence.

Changing a base at a single location (L) in the sequence alters the current predicted as this base passes through the pore's main constriction, but also alters the current before and after the base passes through this main constriction. Predicted current levels were analysed for the set of edited 3.6 Kb lambda sequences to calculate the range of predicted currents at location L+X (offset) when the base at position L is changed. Offsets between-16 and +16 were analysed at each location. The median range of predicted currents at each offset were calculated to give data in the diagrams. Models were centered such that the largest peak, indicative of the CsgG constriction, corresponds to position 0.

### Example 3

The de novo fusion protein sequences designed using Rosetta were analyzed, and sequences for experimental validation were chosen based on the lowest energy score and highest PackStat score (FIG. 2). PSIPRED (e.g., as described by McGuffin L J, Bryson, K, Jones D, Bioinformatics, 16, 404-405, 2000) analysis was conducted to predict fusion protein secondary structure. Residues are shaded according to whether they are predicted to be strand, helix and coil, respectively. Secondary structure analysis of a de novo designed fusion protein (e.g., an elongated CsgF protein) and the mature sequence of wild-type CsgF are shown in FIG. 3A. Structure analysis for de novo designed fusion proteins, ONT1 to ONT10, ONT11 to ONT20, and ONT21 to ONT25 are shown in FIGS. 3B-3C.

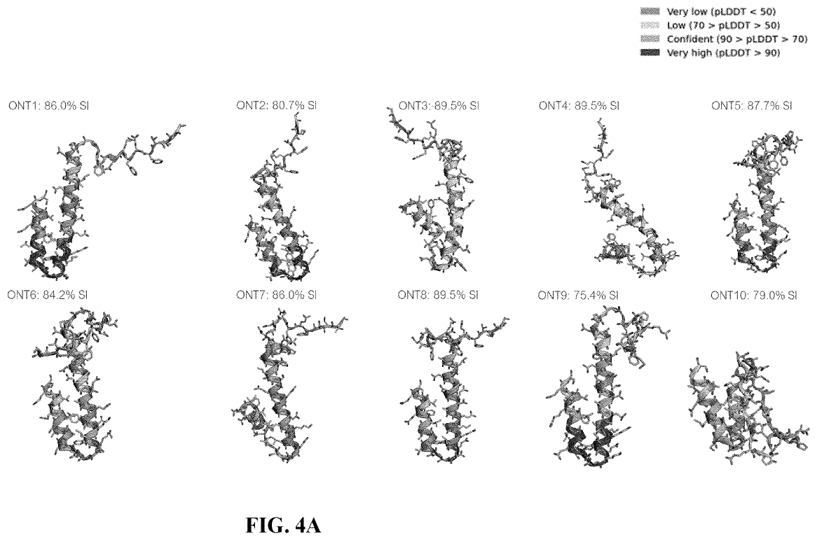

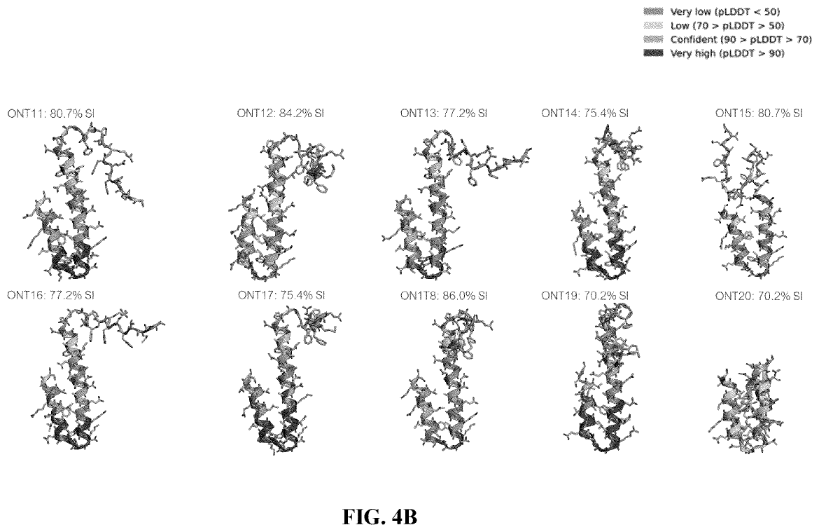

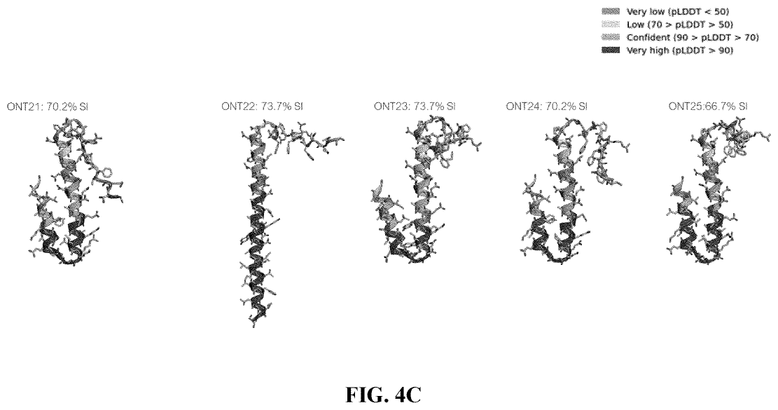

Three-dimensional structures of alternative sequences for de novo designed fusion proteins were also investigated using a protein-folding algorithm. Predicted 3-D structures for de novo designed fusion proteins ONT1 to ONT10, ONT11 to ONT20, and ONT21 to ONT25, are shown in FIGS. 4A-4C. Structures are shaded according to a confidence measure, the predicted local-distance difference test (pLDDT).

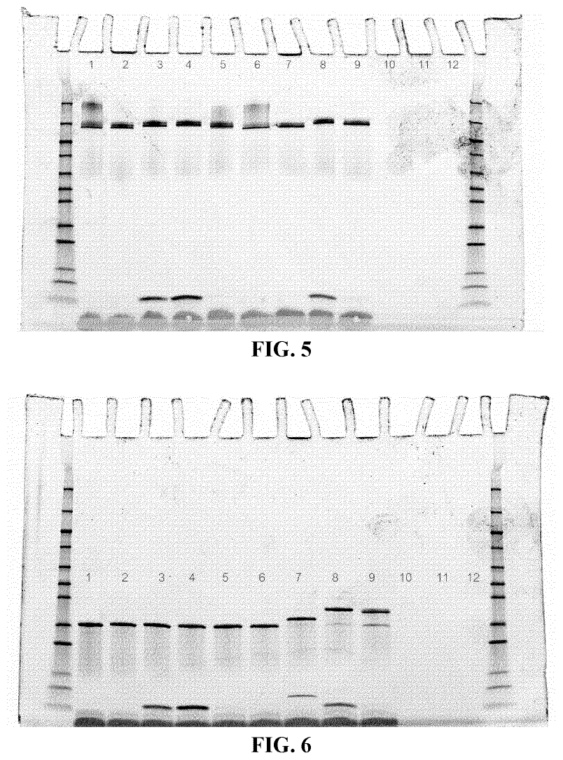

SDS-PAGE gel analysis of CsgG-only pores and CsgG/fusion protein complexes was performed. The complexes either comprised a CsgF-del (S31-F119) control or de novo designed fusion proteins, with or without a maleimide crosslinker (FIG. 5). The complexes comprising the fusion proteins showed a band shift, indicating these samples are nanopore complexes. Note that the samples were not heated prior to loading onto the gel. SDS-PAGE gel analysis of CsgG-only pores and CsgG/fusion protein complexes was also performed. Those complexes either comprised a CsgF-del (S31-F119) control or de novo designed fusion proteins, with or without a maleimide crosslinker (FIG. 6). The pores were broken down to their constituent monomer components upon boiling in the presence of DTT prior to loading onto the gel. Note that no band shifts were observed in the absence of a maleimide crosslink, indicating these bands are comprised of CsgG monomer only. Lane 7 showed a band shift compared to the CsgG-only control, indicating the fusion protein is covalently bound to the CsgG pore due to the presence of the maleimide. Lanes 8 and 9 showed a further band shift due to the increased mass of the fusion proteins. This shows that the fusion protein is covalently bound to the CsgG pore.

Ionic current (pA) versus time(s) as single stranded DNA translocates through CsgG-only pores was measured. Each individual graph corresponds to a single pore inserted into a minION flow cell. The open pore current observed for CsgG-only pores was approximately 180 pA under the applied voltage of −180 mV. Table 1 below shows representative data for the median range, median noise, and median signal to noise ratio (SNR) of protein pore complexes as described by the disclosure.

TABLE 1
Metrics Table

| | Median | Median | |
|---|---|---|---|
| | Range | noise | Median |
| Pore monomer (conjugate) | (pA) | (pA) | SNR |
| CsgG-F56Q | 25.01 | 3.84 | 6.49 |
| CsgG-F56Q/del(S31-F119) | 12.64 | 1.77 | 7.11 |
| CsgG-F56Q/del(S31-F119)-EXT1 | 12.63 | 1.78 | 6.99 |
| CsgG-F56Q/K37R-del(S31- | 12.46 | 1.74 | 7.04 |
| F119)-EXT1 | | | |
| CsgG-F56Q/N24C-K37R- | 15.72 | 2.59 | 5.71 |
| del(S31-F119)-EXT2 | | | |
| CsgG-F56Q-Q153C | 25.62 | 3.53 | 7.26 |
| CsgG-F56Q-Q153C/Mal- | 13.17 | 1.75 | 7.48 |
| Del(S31-F119) | | | |
| CsgG-F56Q-Q153C/Mal-K37R- | 12.85 | 1.77 | 7.25 |
| Del(S31-F119)-EXT1 | | | |
| CsgG-F56Q-Q153C/Mal- | 15.81 | 2.15 | 7.36 |
| N24C/K37R-Del(S31-F119)-EXT2 | | | |

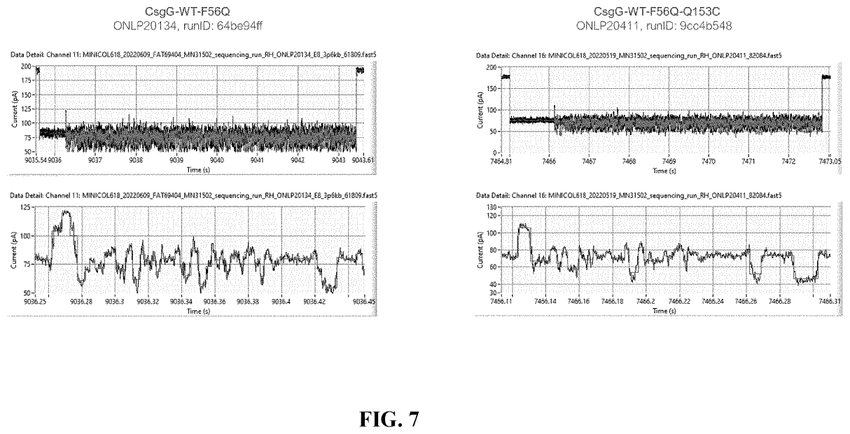

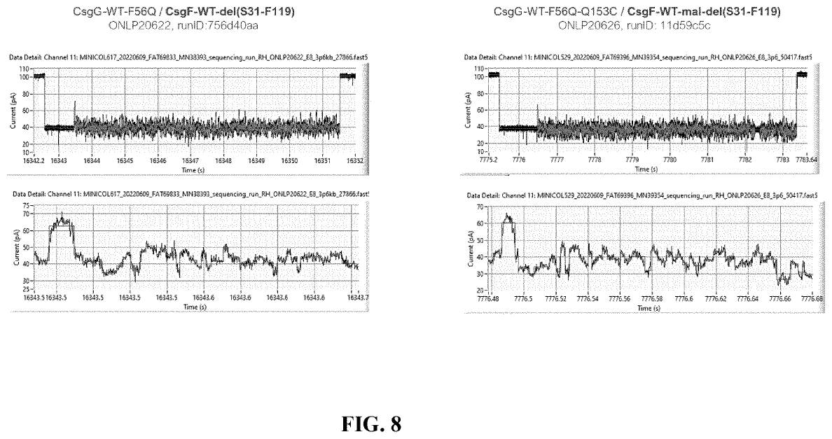

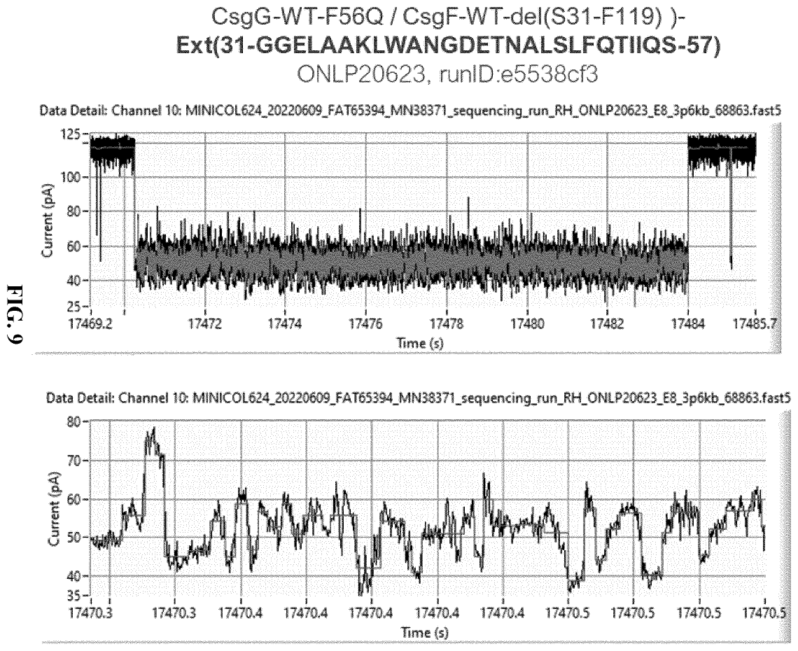

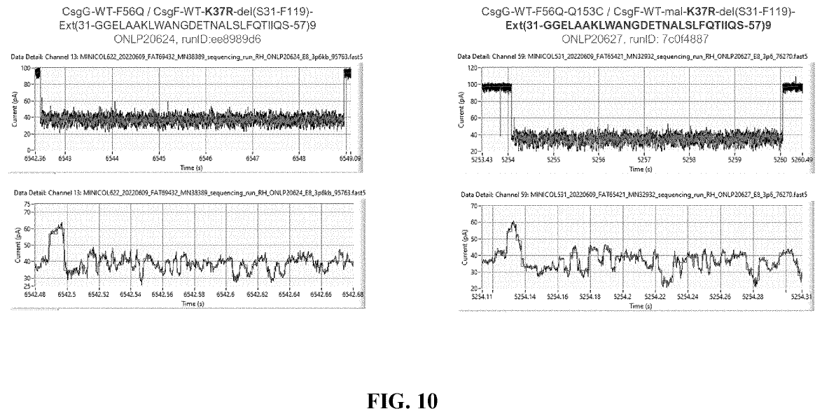

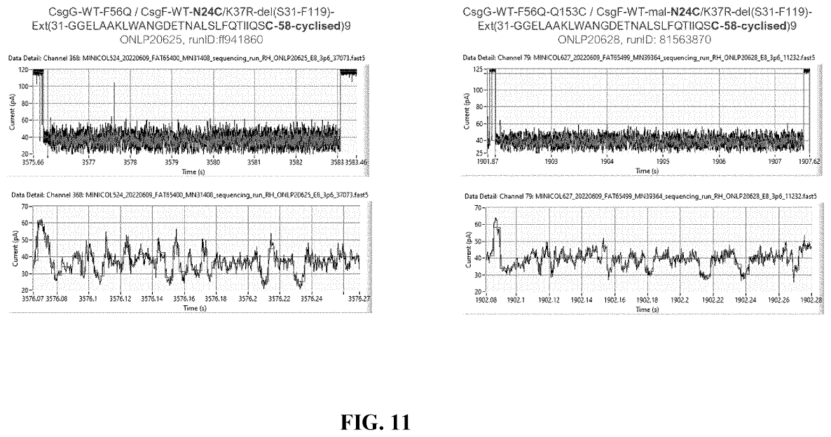

FIGS. 7-11 show representative ionic current (pA) versus time(s) traces as single stranded DNA translocates through CsgG-only pores, CsgG that comprises del (S31-F119) CsgF peptides, or CsgG that comprises de novo designed fusion proteins. The raw current trace is shown in black lines and the event detected signal is shown in red lines. For each pore, the top row shows the full DNA current trace, whilst the bottom row shows a zoomed in view of the first section of the current trace. The open pore current for pores that are CsgG only was observed to be approximately 175-200 pA, and the median current of the DNA squiggle is approximately 75 pA. For pores comprising a CsgF peptide, the open pore current is approximately 90-120 pA, and the median current is approximately 35-50 pA. FIG. 7 shows the trace as DNA translocates through a CsgG-only pore. FIG. 8 shows representative ionic current (pA) versus time(s) traces as single stranded DNA translocates through CsgG that comprises del (S31-F119) CsgF peptides, with (right) or without (left) a maleimide crosslinker. FIG. 9 shows representative ionic current (pA) versus time(s) traces as single stranded DNA translocates through CsgG that comprises a de novo designed fusion protein, ONLP20623, in the absence of a maleimide crosslinker. FIG. 10 shows representative ionic current (pA) versus time(s) traces as single stranded DNA translocates through CsgG that comprises a de novo designed fusion protein, ONLP20624 (without a maleimide crosslink) or ONLP20627 (with a maleimide crosslink). FIG. 11 shows representative ionic current (pA) versus time(s) traces as single stranded DNA translocates through CsgG that comprises a de novo designed fusion protein, ONLP20628 (with a maleimide crosslinker) or ONLP20625 (without a maleimide crosslinker). In some embodiments, the fusion protein comprises 37R residue along with cysteine residues to form an internal disulfide bond within the peptide, i.e. to cyclise the fusion protein.

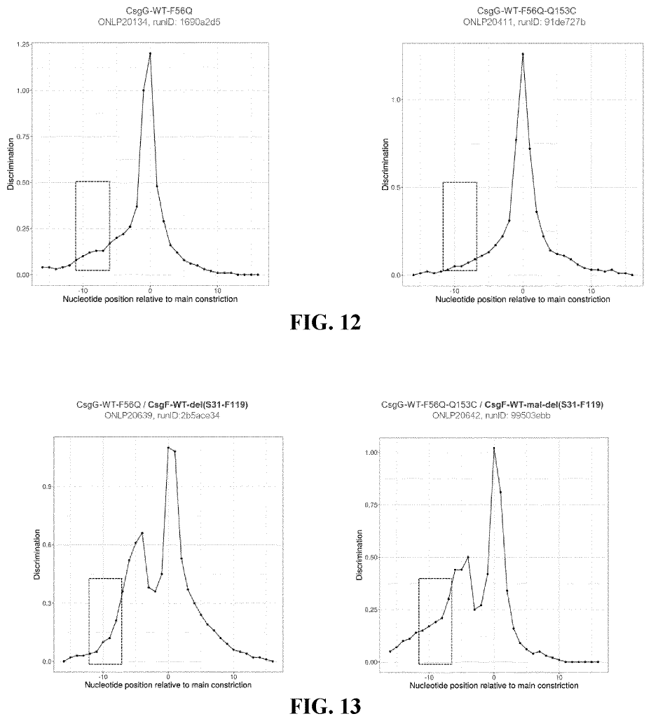

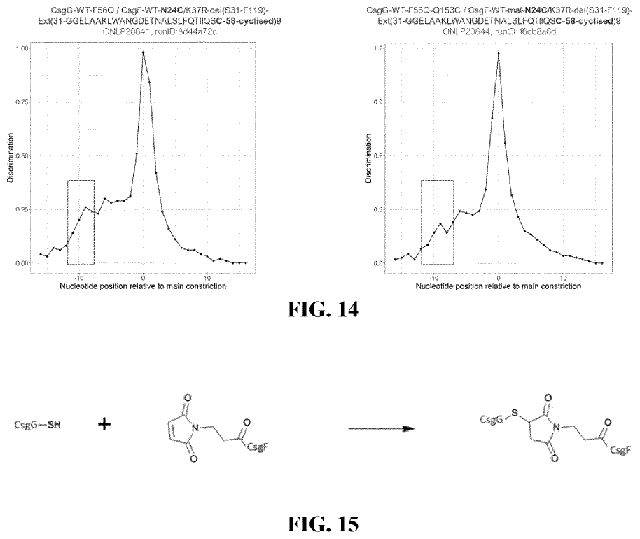

Profiles demonstrating positions within the pore and their contribution to overall changes in ionic current level (“Discrimination”) when a DNA molecule is translocated through the pore were produced. Distances within the pore are measured in nucleotide steps relative to the major constriction. Negative values correspond to positions below the main constriction and positive values correspond to positions above the main constriction (CsgG). Dashed boxes show the region that would be affected by the introduction of a de novo designed fusion protein. FIG. 12 shows representative profiles when a DNA molecule is translocated through CsgG-only pores. CsgG-only pores (plus/minus Q153C) show one major discrimination peak at position 0. FIG. 13 shows representative profiles when a DNA molecule is translocated through a CsgG/CsgF pore. Dashed boxes show the region that would be affected by the introduction of a de novo designed fusion protein. CsgG-CsgF-del (S31-F119) pores with (right) or without (left) a maleimide crosslinker show two discrimination peaks. The major discrimination peak at position 0, as seen in CsgG-only pores, and an additional discrimination peak 4-6 nucleotides below the main constriction (position −4 to −6). This additional region of discrimination has less influence on the ionic current compared to the main discrimination peak at position 0. FIG. 14 shows representative profiles when a DNA molecule is translocated through a CsgG/Fusion Protein (ONLP20641 or ONLP20644) pore. Complexes comprised of CsgG and de novo designed fusion proteins containing K37R with (right) or without (left) a maleimide crosslinker; with cyclisation, show three discrimination peaks. The major discrimination peak at position 0, as seen in CsgG-only pores, and additional peaks at positions −6 and −9. The peak at position −9 corresponds to the expected constriction produced by the de novo designed fusion protein when folded in the correct orientation.

### Example 3

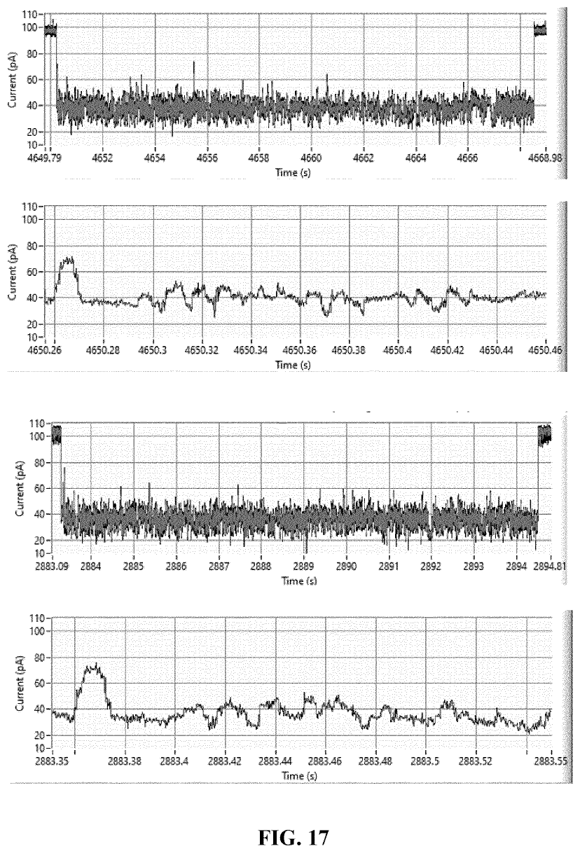

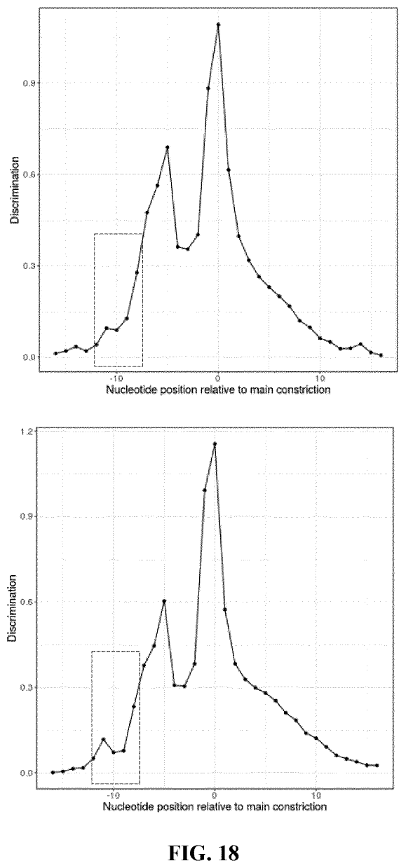

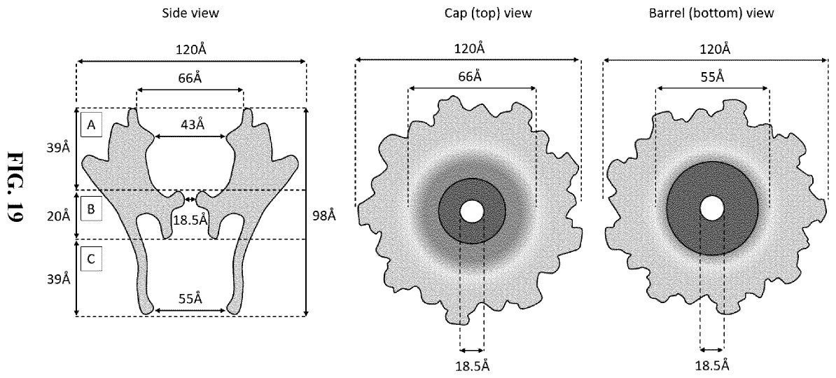

A pore formed from nine of the subunits shown in SEQ ID NO: 61 (with or without a maleimide crosslinker; both without cyclisation) was also tested as described in Example 2. The results are shown in FIGS. 17-18.

REPRESENTATIVE SEQUENCES

> (SEQ ID NO: 1)
CsgF-WT-del(S31-F119))-Ext(31-
GGELAAKLWANGDETNALSLFQTIIQS) (ONLP20623)
GTMTFQFRNPNFGGNPNNGAFLLNSAQAQNGGELAAKLWANGDETNALSLFQTIIQS
> (SEQ ID NO: 2)
CsgF-WT-K37R-del(S31-F119)-Ext(31-
GGELAAKLWANGDETNALSLFQTIIQS) (ONLP20624)
GTMTFQFRNPNFGGNPNNGAFLLNSAQAQNGGELAARLWANGDETNALSLFQTIIQS
> (SEQ ID NO: 3)
CsgF-WT-N24C/K37R-del(S31-F119)-Ext(31-
GGELAAKLWANGDETNALSLFQTIIQSC) (ONLP20625)
GTMTFQFRNPNFGGNPNNGAFLLCSAQAQNGGELAARLWANGDETNALSLFQTIIQSC
> (SEQ ID NO: 4)
Mat-CsgF-Eco-(WT-Del(S31-F119)-Ext(31-
AGELAKKLWENGNVNQALSLFQTVIQS) (ONLZ19432, DGLONT76)
GTMTFQFRNPNFGGNPNNGAFLLNSAQAQNAGELAKKLWENGNVNQALSLFQTVIQS
> (SEQ ID NO: 5)
Mat-CsgF-Eco-(WT-K36R/K37R-Del(S31-F119)-Ext(31-
AGELAKKLWENGNVNQALSLFQTVIQS) (ONLZ19431)
GTMTFQFRNPNFGGNPNNGAFLLNSAQAQNAGELARRLWENGNVNQALSLFQTVIQS
> (SEQ ID NO: 6)
Mat-CsgF-Eco-(WT-N24C/K36R/K37R-Del(S31-F119)-Ext(31-
AGELARRLWENGNVNQALSLFQTVIQSC) (ONLZ19781)
GTMTFQFRNPNFGGNPNNGAFLLCSAQAQNAGELARRLWENGNVNQALSLFQTVIQSC
>(SEQ ID NO: 7)
ONT113_2
GTMTFQFRNPNFGGNPNNGAFLLNSAQAQNAAELAAKLWANADETNALSLFQTIIQS
>(SEQ ID NO: 8)
ONT113_3
GTMTFQFRNPNFGGNPNNGAFLLNSAQAQNAAELAAKLWANADETNALSLFQTLIQS
> (SEQ ID NO: 9)
ONT1
GTMTFQFRNPNFGGNPNNGAFLLNSAQAQNAGELAAKLFKKGDLTNALSLFQTVIQS
> (SEQ ID NO: 10)
ONT2
GTMTFQFRNPNFGGNPNNGAFLLNSAQAQNAGELVEKLFKNGDWTNAISIFQTVIQS
> (SEQ ID NO: 11)
ONT 3
GTMTFQFRNPNFGGNPNNGAFLLNSAQAQNAGELAEKLWRNGDETNALSLFQTVIQS
> (SEQ ID NO: 12)
ONT 4
GTMTFQFRNPNFGGNPNNGAFLLNSAQAQNAGELAEKLWKNGDETNALSLFQTVIQS
> (SEQ ID NO: 13)
ONT 5
GTMTFQFRNPNFGGNPNNGAFLLNSAQAQNAGELAKKLWENGDETNALSLFQTVVQS
> (SEQ ID NO: 14)
ONT 6
GTMTFQFRNPNFGGNPNNGAFLLNSAQAQNAGELAEKLWRNGNESDALSLFQTVIQS
> (SEQ ID NO: 15)
ONT 7
GTMTFQFRNPNFGGNPNNGAFLLNSAQAQNAGELAKKLFENGDKTNALSLFQTVIQS
> (SEQ ID NO: 16)
ONT 8
GTMTFQFRNPNFGGNPNNGAFLLNSAQAQNAGELAKKLWENGDETNALSLFQTVIQS
> (SEQ ID NO: 17)
ONT 9
GTMTFQFRNPNFGGNPNNGAFLLNSAQAQNAGELAKKLWEKGNSEDALALFRTVVQS
> (SEQ ID NO: 18)
ONT 10
GTMTFQFRNPNFGGNPNNGAFLLNSAQAQNAGELAKKLFDNGDMENAMKLFQTVIAS
> (SEQ ID NO: 19)
ONT 11
GTMTFQFRNPNFGGNPNNGAFLLNSAQAQNAGELAEKLWRNGDKDRALALFRTVIQS
> (SEQ ID NO: 20)
ONT 12
GTMTFQFRNPNFGGNPNNGAFLLNSAQAQNAGELADKLWKNGDKDRALSLFQTVIQS
> (SEQ ID NO: 21)
ONT 13
GTMTFQFRNPNFGGNPNNGAFLLNSAQAQNAGELAKKLFDNGDMDRALALFRTVIAS
> (SEQ ID NO: 22)
ONT 14
GTMTFQFRNPNFGGNPNNGAFLLNSAQAQNAGELAKKLFDNGNEEDALALFRTVVAS
> (SEQ ID NO: 23)
ONT 15
GTMTFQFRNPNFGGNPNNGAFLLNSAQAQNAGELAAKLWKKGDEENALKLFRTVVTS
> (SEQ ID NO: 24)
ONT 16
GTMTFQFRNPNFGGNPNNGAFLLNSAQAQNAGELAAKLFKNGNMEDALKLFRTVIAS
> (SEQ ID NO: 25)
ONT 17
GTMTFQFRNPNFGGNPNNGAFLLNSAQAQNAGKVAAILWKNGNKSDALSLFQTVVTS
> (SEQ ID NO: 26)
ONT 18
GTMTFQFRNPNFGGNPNNGAFLLNSAQAQNAGELAAKLFKNGDLTNALSLFQTVVQS
> (SEQ ID NO: 27)
ONT 19
GTMTFQFRNPNFGGNPNNGAFLLNSAQAQNAGELGLKLLRKGDVETALTLFAQVISG
> (SEQ ID NO: 28)
ONT 20
GTMTFQFRNPNFGGNPNNGAFLLNSAQAQNAGELGLKLILKGDLETALKLFAIVIAG
> (SEQ ID NO: 29)
ONT 21
GTMTFQFRNPNFGGNPNNGAFLLNSAQAQNAGELGLKLLRKGDVETALKLFAIVIAG
> (SEQ ID NO: 30)
ONT 22
GTMTFQFRNPNFGGNPNNGAFLLNSAQAQNAGELAKKLYENGLIELALMLFALVIAS
> (SEQ ID NO: 31)
ONT 23
GTMTFQFRNPNFGGNPNNGAFLLNSAQAQNAGELYKKLWDNGEVDKALDLFAKIIAG
> (SEQ ID NO: 32)
ONT 24
GTMTFQFRNPNFGGNPNNGAFLLNSAQAQNAGELGKKLIEKGDLETALKLFAIVIAG
> (SEQ ID NO: 33)
ONT 25
GTMTFQFRNPNFGGNPNNGAFLLNSAQAQNAGEIALRLLKNGKEEEALKTLLVTIAG
> (SEQ ID NO: 34)
ONT26
GTMTFQFRNPNFGGNPNNGAFLLNSAQAQNAGELAAKLWKKGDETNALSLFQTVVTS
> (SEQ ID NO: 35)
ONT27
GTMTFQFRNPNFGGNPNNGAFLLNSAQAQNAGKVAAILWKNGNKSDALSLFQTVVTS
> (SEQ ID NO: 36)
ONT28
GTMTFQFRNPNFGGNPNNGAFLLNSAQAQNAGELAKKLWEKGDETNALSLFQTVVTS
> (SEQ ID NO: 37)
ONT29
GTMTFQFRNPNFGGNPNNGAFLLNSAQAQNAGDLAAKLWKKGDETNALSLFQTVVTS
> (SEQ ID NO: 38)
ONT30
GTMTFQFRNPNFGGNPNNGAFLLNSAQAQNAGELAAKLWKNGNSSDALSLFQTVVTS
> (SEQ ID NO: 39)
ONT31
GTMTFQFRNPNFGGNPNNGAFLLNSAQAQNAGELAKKLWEKGDETNALSLFQTVVTS
> (SEQ ID NO: 40)
ONT32
GTMTFQFRNPNFGGNPNNGAFLLNSAQAQNAGELAKKLWEKGDSSNALSLFQTVVTS
> (SEQ ID NO: 41)
ONT33
GTMTFQFRNPNFGGNPNNGAFLLNSAQAQNAGDLAAKLWKNGDETNALSLFQTVVTS
> (SEQ ID NO: 42)
ONT34
GTMTFQFRNPNFGGNPNNGAFLLNSAQAQNAGELAAKLFKNGDLTNALSLFQTVVQS
> (SEQ ID NO: 43)
ONT35
GTMTFQFRNPNFGGNPNNGAFLLNSAQAQNAGELAAKLWKKGDETNALSLFQTVVTS
> (SEQ ID NO: 44)
ONT36
GTMTFQFRNPNFGGNPNNGAFLLNSAQAQNAGELAAKLFNSGDLDRALALFRTVVTS
> (SEQ ID NO: 45)
ONT37
GTMTFQFRNPNFGGNPNNGAFLLNSAQAQNAGKVAKELYDNGDEKWALLLFRTVVTS
> (SEQ ID NO: 46)
ONT38
GTMTFQFRNPNFGGNPNNGAFLLNSAQAQNAGKVAAELYKNGDEKNALLLFRTVVAS
>(SEQ ID NO: 47)
ONT39
GTMTFQFRNPNFGGNPNNGAFLLNSAQAQNAGELAAKLFKNGDMENALALFRTVVTS
>(SEQ ID NO: 48)
ONT40
GTMTFQFRNPNFGGNPNNGAFLLNSAQAQNAGELAKKLWEKGNSEDALALFRTVVQS
> (SEQ ID NO: 49)
ONT41
GTMTFQFRNPNFGGNPNNGAFLLNSAQAQNAGELAAKLFNKGDEDRALALFRTVVQS
> (SEQ ID NO: 50)
ONT42
GTMTFQFRNPNFGGNPNNGAFLLNSAQAQNAGELAAKLWKNGDEENALALFRTVVTS
> (SEQ ID NO: 51)
ONT43
GTMTFQFRNPNFGGNPNNGAFLLNSAQAQNAGELAEKLWRSGDADRALALFRTVVTS
> (SEQ ID NO: 52)
ONT44
GTMTFQFRNPNFGGNPNNGAFLLNSAQAQNAGELAAKLWKNGNEEDALALFRTVVTS
> (SEQ ID NO: 53)
ONT45
GTMTFQFRNPNFGGNPNNGAFLLNSAQAQNAGELAAKLFNNGDEDRALALFRTVVQS
> (SEQ ID NO: 54)
ONT46
GTMTFQFRNPNFGGNPNNGAFLLNSAQAQNAGELAAKLWKKGDEDRALALFRTVVTS
> (SEQ ID NO: 55)
ONT47
GTMTFQFRNPNFGGNPNNGAFLLNSAQAQNAGELAAKLFNSGDEDRALALFRTVVQS
> (SEQ ID NO: 56)
ONT48
GTMTFQFRNPNFGGNPNNGAFLLNSAQAQNAGELAAKLYNNGDLDRADATFRTVVQS
> (SEQ ID NO: 57)
ONT49
GTMTFQFRNPNFGGNPNNGAFLLNSAQAQNAGELAKKLWENGNEEDALALFRTVVTS
> (SEQ ID NO: 58)
ONT50
GTMTFQFRNPNFGGNPNNGAFLLNSAQAQNAGEIAKQLWEKGDESSAITVATIVLSS
>(SEQ ID NO: 59)
Wild-type E. coli CsgG protein monomer (no signal sequence)
CLTAPPKEAARPTLMPRAQSYKDLTHLPAPTGKIFVSVYNIQDETGQFKPYPASNFSTAV
PQSATAMLVTALKDSRWFIPLERQGLQNLLNERKIIRAAQENGTVAINNRIPLQSLTAAN
IMVEGSIIGYESNVKSGGVGARYFGIGADTQYQLDQIAVNLRVVNVSTGEILSSVNTSKT
ILSYEVQAGVFRFIDYQRLLEGEVGYTSNEPVMLCLMSAIETGVIFLINDGIDRGLWDLQ
NKAERQNDILVKYRHMSVPPES
>(SEQ ID NO: 60)
Residues 1-30 of CsgF pep tide
GTMTFQFRNPNFGGNPNNGAFLLNSAQAQN
>(SEQ ID NO: 61)
CsgF-WT-del(S31-F119)-Ext(31-AGILAAQLWNNGDYDRALSLFIAVVQS-57)
GTMTFQFRNPNFGGNPNNGAFLLNSAQAQNAGILAAQLWNNGDYDRALSLFIAVVQS

What is claimed is:

1. A protein nanopore complex comprising:
(a) a CsgG nanopore comprising a lumen; and
(b) a fusion polypeptide comprising a first portion comprising a CsgF protein and a second portion comprising a helix-forming auxiliary protein, wherein the fusion protein is attached to the nanopore.

2. The protein nanopore complex of claim 1, wherein the first portion of the fusion protein is attached to the CsgG nanopore.

3. The protein nanopore complex of claim 1 or 2, wherein the first portion of the fusion protein is positioned inside the lumen of the CsgG nanopore.

4. The protein nanopore complex of any one of claims 1 to 3, wherein the first portion of the fusion protein extends outside of the lumen of the CsgG nanopore.

5. The protein nanopore complex of any one of claims 1 to 4, wherein the first portion forms a first constriction region in the lumen of the CsgG nanopore.

6. The protein nanopore complex of claim 5, wherein the second portion forms a second constriction region.

7. The protein nanopore complex of any one of claims 1 to 6, wherein the CsgG nanopore further comprises a constriction region.

8. The protein nanopore complex of any one of claims 1 to 7, wherein the second portion is not attached to the CsgG nanopore.

9. The protein nanopore complex of any one of claims 1 to 8, wherein the second portion comprises one or more alpha-helices.

10. The protein nanopore complex of any one of claims 1 to 9, wherein each of the alpha-helices comprises between 0 and 15 alpha-helical turns.

11. The protein nanopore complex of any one of claims 1 to 10, wherein the second portion comprises a first alpha-helix comprising one to four alpha-helical turns, and a second alpha-helix comprising three to six alpha-helical turns.

12. The protein nanopore complex of claim 11, wherein the second alpha-helix packs against the first alpha-helix.

13. The protein nanopore complex of any one of claims 9 to 12, wherein the second portion comprises between 1 and 55 amino acid residues.

14. The protein nanopore complex of any one of claims 6 to 13, wherein the distance between the first constriction region and second constriction region ranges from about 5 Å to about 80 Å.

15. The protein nanopore complex of any one of claims 1 to 14, wherein the protein nanopore complex has an axial length greater than 90 Å, optionally wherein the axial length ranges from about 95 Å to about 160 Å.

16. The protein nanopore complex of any one of claims 1 to 15, wherein the fusion protein is attached to the nanopore by a linker.

17. The protein nanopore complex of claim 16, wherein the linker comprises a bond, a peptide linker, or a chemical linker.

18. The protein nanopore complex of claim 16 or 17, wherein the linker comprises a bond formed by a Sulfur(VI) fluoride exchange (SuFEx) reaction.

19. The protein nanopore complex of claim 16 or 17, wherein the linker comprises one or more maleimide molecules.

20. The protein nanopore complex of any one of claims 1 to 19, wherein the fusion protein is cyclised.

21. The protein nanopore complex of claim 20, wherein the cyclisation comprises one or more side-chain to side-chain cyclisation bonds.

22. The protein nanopore complex of claim 21, wherein at least one of the side-chain to side-chain cyclisation bonds is a disulfide bond.

23. A protein nanopore complex comprising:
(a) a CsgG nanopore comprising a lumen and a first constriction region formed within the lumen of the nanopore; and
(b) a fusion protein comprising a first portion comprising a CsgF protein and a second portion comprising a helix-forming auxiliary protein, wherein the fusion protein is attached to the nanopore.

24. The protein nanopore complex of claim 23, wherein the first portion of the fusion protein is attached to the CsgG nanopore.

25. The protein nanopore complex of claim 23 or 24, wherein the first portion of the fusion protein is positioned inside the lumen of the CsgG nanopore.

26. The protein nanopore complex of any one of claims 23 to 25, wherein the second portion of the fusion protein is positioned outside the lumen of the CsgG nanopore.

27. The protein nanopore complex of any one of claims 23 to 26, wherein the first portion forms a second constriction region in the lumen of the CsgG nanopore.

28. The protein nanopore complex of claim 27, wherein the second portion forms a third constriction region in the lumen of the CsgG nanopore.

29. The protein nanopore complex of any one of claims 23 to 28, wherein the second portion is not attached to the CsgG nanopore.

30. The protein nanopore complex of any one of claims 23 to 29, wherein the second portion comprises one or more alpha-helices.

31. The protein nanopore complex of claim 30, wherein each of the alpha-helices comprises between 0 and 15 alpha-helical turns.

32. The protein nanopore complex of any one of claims 23 to 31, wherein the second portion comprises between 1 and 55 amino acid residues.

33. The protein nanopore complex of any one of claims 23 to 32, wherein the fusion protein is cyclised.

34. The protein nanopore complex of claim 33, wherein the cyclisation comprises one or more side-chain to side-chain cyclisation bonds.

35. The protein nanopore complex of claim 34, wherein at least one of the side-chain to side-chain cyclisation bonds is a disulfide bond.

36. A protein nanopore complex comprising:
(a) a CsgG nanopore comprising a lumen and a first constriction region formed within the lumen of the nanopore;
(b) a first auxiliary protein attached to the CsgG nanopore and forming a second constriction region in the lumen of the nanopore; and
(c) a second auxiliary protein attached to the CsgG nanopore or the first auxiliary protein, and forming a third constriction region.

37. The protein nanopore complex of claim 36, wherein the first auxiliary protein is positioned inside the lumen of the CsgG nanopore.

38. The protein nanopore complex of claim 36 or 37, wherein the first auxiliary protein comprises a CsgF protein or peptide.

39. The protein nanopore complex of any one of claims 36 to 38, wherein the second auxiliary protein comprises one or more alpha-helices.

40. The protein nanopore complex of claim 39, wherein each of the one or more alpha-helices comprises between 0 and 15 alpha-helical turns.

41. The protein nanopore complex of claim 39 or 40, wherein the second auxiliary protein comprises two alpha-helices.

42. The protein nanopore complex of claim 41, wherein one of the alpha-helices comprises between 1 and 6 alpha-helical turns.

43. The protein nanopore complex of claim 41 or 42, wherein one of the alpha-helices comprises between 1 and 10 alpha-helical turns.

44. The protein nanopore complex of any one of claims 41 to 43, wherein one of the alpha-helices comprises three alpha-helical turns, and the other alpha-helix comprises three or four alpha-helical turns.

45. The protein nanopore complex of any one of claims 36 to 44, wherein the second auxiliary protein comprises at least one alpha helix that packs against an alpha-helix of the first auxiliary protein.

46. The protein nanopore complex of any one of claims 36 to 45, wherein the second auxiliary protein comprises between 1 and 55 amino acid residues.

47. The protein nanopore complex of any one of claims 36 to 46, wherein the distance between the first constriction and second constriction ranges from about 10 Å to about 80 Å.

48. The protein nanopore complex of any one of claims 36 to 47, wherein the distance between the second constriction and third constriction ranges from about 5 Å to about 80 Å.

49. The protein nanopore complex of any one of claims 36 to 48, wherein the protein nanopore complex has an axial length greater than 90 Å, optionally wherein the axial length ranges from about 95 Å to about 160 Å.

50. The protein nanopore complex of any one of claims 36 to 49, wherein the first auxiliary protein and the second auxiliary protein are attached by a linker.

51. The protein nanopore complex of claim 50, wherein the linker comprises a bond, a peptide linker, or a chemical linker.

52. The protein nanopore complex of claim 50 or 51, wherein the linker comprises a bond formed by a Sulfur (VI) fluoride exchange (SuFEx) reaction.

53. The protein nanopore complex of claim 50 or 51, wherein the linker comprises one or more maleimide molecules.

54. The protein nanopore complex of any one of claims 36 to 53, wherein the first auxiliary protein and the second auxiliary protein comprise one or more side-chain to side-chain cyclisation bonds.

55. The protein nanopore complex of claim 54, wherein at least one of the side-chain to side-chain cyclisation bonds is a disulfide bond.

56. A system for characterising a target analyte, the system comprising the protein nanopore complex of any one of claims 1 to 55 inserted into a membrane.

57. The system of claim 56, further comprising: an electrically-conductive solution in contact with the protein nanopore complex, electrodes providing a voltage potential across the membrane, and a measurement system for measuring the current through the protein nanopore complex.

58. A method for characterising a target analyte, the method comprising the steps of: (a) contacting a system according to claim 56 with the target analyte; (b) applying a potential across the membrane such that the target analyte moves with respect to the lumen formed by the protein nanopore complex; and (c) taking one or more measurements as the target analyte moves with respect to the lumen, thereby characterising the target analyte.

59. The method of claim 58, wherein the target analyte comprises a target polynucleotide.

60. The method according to claim 58 or 59, wherein step (c) comprises measuring the current passing through the continuous channel, wherein the current is indicative of the presence and/or one or more characteristics of the target analyte and thereby detecting and/or characterising the target analyte.

61. The method of any one of claims 58 to 60, wherein the target analyte is a polynucleotide and nucleotides in the polynucleotide interact with the first, second, and, optionally, third constriction regions within the lumen and wherein each of the first, second, and, optionally, third constriction regions is capable of discriminating between different nucleotides, such that the overall current passing through the lumen is influenced by the interactions between each of the first, second, and third constriction regions and the nucleotides located at each of the regions.
